SEQUENCE LISTING (232 sequences)

SEQ ID NO: 1 - DNA, 1638 bp, Cannabis sativa
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcgtgagttgggtattaaaaaaactgattgcaaagaatttagctggattgatacaaccatcttctacaatggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 2 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFRELGIKKTDCKEFSWIDTTIFYNGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 3 - DNA, 23 bp, Cannabis sativa
ttacaacaccattgtagaagatg

SEQ ID NO: 4 - DNA, 23 bp, Cannabis sativa
tacacttttgacgcctttgatgc

SEQ ID NO: 5 - DNA, 23 bp, Cannabis sativa
gtgctcgcaaattccacttgcaa

SEQ ID NO: 6 - DNA, 23 bp, Cannabis sativa
taagcactacaggggcacgtacg

SEQ ID NO: 7 - DNA, 23 bp, Cannabis sativa
aaaataaatacttttttttttgt

SEQ ID NO: 8 - DNA, 23 bp, Cannabis sativa
ttaggggtggactatattccaca

SEQ ID NO: 9 - DNA, 23 bp, Cannabis sativa
attaaagaatgtatgcctttgct

SEQ ID NO: 10 - DNA, 23 bp, Cannabis sativa
gatacaaataatattggacagaa

SEQ ID NO: 11 - DNA, 23 bp, Cannabis sativa
gtagtcaagttcagattcaaagc

SEQ ID NO: 12 - DNA, 23 bp, Cannabis sativa
gattactttactaagtggactga

SEQ ID NO: 13 - DNA, 23 bp, Cannabis sativa
tgaccttcttcttcaacctggcc

SEQ ID NO: 14 - DNA, 23 bp, Cannabis sativa
atgtaatgggccagcatctcctt

SEQ ID NO: 15 - DNA, 23 bp, Cannabis sativa
actaccaggacgtacatcatcct

SEQ ID NO: 16 - DNA, 23 bp, Cannabis sativa
tctaagatcaactagtgatattg

SEQ ID NO: 17 - DNA, 23 bp, Cannabis sativa
ggaaggataatctgtatgttatt

SEQ ID NO: 18 - DNA, 23 bp, Cannabis sativa
gtagtgattttggagaagtgtaa

SEQ ID NO: 19 - DNA, 23 bp, Cannabis sativa
cactgttgactgaaaataagcta

SEQ ID NO: 20 - DNA, 23 bp, Cannabis sativa
ttgattaactgtcttatttagtg

SEQ ID NO: 21 - DNA, 23 bp, Cannabis sativa
aaatacataaggaagccaaatat

SEQ ID NO: 22 - DNA, 23 bp, Cannabis sativa
attataacaatggataactaatg

SEQ ID NO: 23 - DNA, 23 bp, Cannabis sativa
tcttttcttctcttaaaattttg

SEQ ID NO: 24 - DNA, 23 bp, Cannabis sativa
tcttgttcatagtggttttgaca

SEQ ID NO: 25 - DNA, 23 bp, Cannabis sativa
cacaaccgaagaggtcaatacca

SEQ ID NO: 26 - DNA, 23 bp, Cannabis sativa
tgtaaaacccataatggagatcg

SEQ ID NO: 27 - DNA, 23 bp, Cannabis sativa
taacgtgataaaattatatgatc

SEQ ID NO: 28 - DNA, 23 bp, Cannabis sativa
acacagatatcgaggaaatcctt

SEQ ID NO: 29 - DNA, 23 bp, Cannabis sativa
gtattatatttatccaggatctt

SEQ ID NO: 30 - DNA, 23 bp, Cannabis sativa
gacaagaaatttattaccagtat

SEQ ID NO: 31 - DNA, 23 bp, Cannabis sativa
tgcagtctttgttgcttcagtct

SEQ ID NO: 32 - DNA, 23 bp, Cannabis sativa
cattagtgtaaatccaataacat

SEQ ID NO: 33 - DNA, 23 bp, Cannabis sativa
atctagatcctagtaaaataatt

SEQ ID NO: 34 - DNA, 23 bp, Cannabis sativa
tataaatcaaatagacaaacaat

SEQ ID NO: 35 - DNA, 23 bp, Cannabis sativa
tgctttaggcaatatattgggat

SEQ ID NO: 36 - DNA, 22 bp, Cannabis sativa
taagtcttataagtcgccacca

SEQ ID NO: 37 - DNA, 23 bp, Cannabis sativa
ctgtaaaaaaggaaattgcttag

SEQ ID NO: 38 - DNA, 23 bp, Cannabis sativa
tgatgcattggaaaagttggcat

SEQ ID NO: 39 - DNA, 23 bp, Cannabis sativa
atctggccgacaactccctctct

SEQ ID NO: 40 - DNA, 23 bp, Cannabis sativa
tgaaataaaacgtggcagctctg

SEQ ID NO: 41 - DNA, 23 bp, Cannabis sativa
gggaatattgaaaaaataatttt

SEQ ID NO: 42 - DNA, 23 bp, Cannabis sativa
catgatatttttaagtcaaaaat

SEQ ID NO: 43 - DNA, 23 bp, Cannabis sativa
aaaccctagtagcatgtccaaaa

SEQ ID NO: 44 - DNA, 23 bp, Cannabis sativa
ccattggctcctagatgcttgat

SEQ ID NO: 45 - DNA, 23 bp, Cannabis sativa
acgccaactttggtatccatgat

SEQ ID NO: 46 - DNA, 23 bp, Cannabis sativa
ataaaaacgataatttatatgtt

SEQ ID NO: 47 - DNA, 23 bp, Cannabis sativa
tatcacctgttgatcctagatca

SEQ ID NO: 48 - DNA, 23 bp, Cannabis sativa
aggtactattagttttaaattgt

SEQ ID NO: 49 - DNA, 23 bp, Cannabis sativa
aaattaataaagcaaaataaaga

SEQ ID NO: 50 - DNA, 23 bp, Cannabis sativa
atagtagaggggattagaatcca

SEQ ID NO: 51 - DNA, 23 bp, Cannabis sativa
aatggatccatagataaatattt

SEQ ID NO: 52 - DNA, 23 bp, Cannabis sativa
cacaaaacatgcaaaattatgaa

SEQ ID NO: 53 - DNA, 23 bp, Cannabis sativa
tcaatatcggtcaagtgcaatgt

SEQ ID NO: 54 - DNA, 23 bp, Cannabis sativa
cttttaggtcagggtgatacgta

SEQ ID NO: 55 - DNA, 23 bp, Cannabis sativa
ttcttcgttgagtccacgcataa

SEQ ID NO: 56 - DNA, 23 bp, Cannabis sativa
cacgttttgatgaggttccatct

SEQ ID NO: 57 - DNA, 23 bp, Cannabis sativa
gtcctgtttaggcgagagtcaag

SEQ ID NO: 58 - DNA, 23 bp, Cannabis sativa
aataaatcatgagaaccatgcaa

SEQ ID NO: 59 - DNA, 23 bp, Cannabis sativa
tatttatttttcgaaaatttaaa

SEQ ID NO: 60 - DNA, 23 bp, Cannabis sativa
tatattaataagtaaatctcatt

SEQ ID NO: 61 - DNA, 23 bp, Cannabis sativa
taagagttgttcactgagagctg

SEQ ID NO: 62 - DNA, 23 bp, Cannabis sativa
aaggtttacaagcacaaccctat

SEQ ID NO: 63 - DNA, 23 bp, Cannabis sativa
gaataaggaatcctcatctcctt

SEQ ID NO: 64 - DNA, 23 bp, Cannabis sativa
atactccttcagttgtctggatg

SEQ ID NO: 65 - DNA, 23 bp, Cannabis sativa
ctcataccaatatgtttactcat

SEQ ID NO: 66 - DNA, 23 bp, Cannabis sativa
ataatctgaactcgaaggagaag

SEQ ID NO: 67 - DNA, 23 bp, Cannabis sativa
tgtaagtcttatacagtcgccac

SEQ ID NO: 68 - DNA, 23 bp, Cannabis sativa
tactccaagtgtaagtatacttc

SEQ ID NO: 69 - DNA, 23 bp, Cannabis sativa
taaattgataactaatcagatta

SEQ ID NO: 70 - DNA, 23 bp, Cannabis sativa
tttcctgttccctggtttatggc

SEQ ID NO: 71 - DNA, 23 bp, Cannabis sativa
cctcccctctggccaagaagtct

SEQ ID NO: 72 - DNA, 23 bp, Cannabis sativa
acgatgatgtatcgtgctgcctt

SEQ ID NO: 73 - DNA, 23 bp, Cannabis sativa
ataatcttaccatacagggattt

SEQ ID NO: 74 - DNA, 23 bp, Cannabis sativa
tcgtctttcagtgctactgctcg

SEQ ID NO: 75 - DNA, 23 bp, Cannabis sativa
ccattgctgtgtggcctctaaca

SEQ ID NO: 76 - DNA, 23 bp, Cannabis sativa
gttttggagttgaaattctgcat

SEQ ID NO: 77 - DNA, 23 bp, Cannabis sativa
cattcaaatacacattaatataa

SEQ ID NO: 78 - DNA, 23 bp, Cannabis sativa
ggctgaataatctaaagtggcca

SEQ ID NO: 79 - DNA, 23 bp, Cannabis sativa
gctcaactcaacggatcactaat

SEQ ID NO: 80 - DNA, 23 bp, Cannabis sativa
tagactggatgctgcacgccatc

SEQ ID NO: 81 - DNA, 23 bp, Cannabis sativa
ctgcaagccaaatttcttggagc

SEQ ID NO: 82 - DNA, 23 bp, Cannabis sativa
gttgttttatgtcgcgaacaaca

SEQ ID NO: 83 - DNA, 23 bp, Cannabis sativa
agagacagaaaaagaagaccctt

SEQ ID NO: 84 - DNA, 23 bp, Cannabis sativa
aaattggggctatgctgatattt

SEQ ID NO: 85 - DNA, 23 bp, Cannabis sativa
attctaattatgtttttgaatta

SEQ ID NO: 86 - DNA, 23 bp, Cannabis sativa
tagctaatctgcggccgacccat

SEQ ID NO: 87 - DNA, 23 bp, Cannabis sativa
aatggtgagcccttaatgcctcc

SEQ ID NO: 88 - DNA, 23 bp, Cannabis sativa
gtttcactaacaggagttgtccc

SEQ ID NO: 89 - DNA, 23 bp, Cannabis sativa
taaagcaaatgagtgtctcccat

SEQ ID NO: 90 - DNA, 23 bp, Cannabis sativa
tgctcaaatcccagagaataaca

SEQ ID NO: 91 - DNA, 23 bp, Cannabis sativa
atatattgtttattgagaggata

SEQ ID NO: 92 - DNA, 23 bp, Cannabis sativa
aggctcaatcggtccaaagaaaa

SEQ ID NO: 93 - DNA, 23 bp, Cannabis sativa
tgatacatttcagtggttcccga

SEQ ID NO: 94 - DNA, 23 bp, Cannabis sativa
atcgttttgcttgaaattttgtc

SEQ ID NO: 95 - DNA, 23 bp, Cannabis sativa
caagggcaaacaggtaattacaa

SEQ ID NO: 96 - DNA, 23 bp, Cannabis sativa
aaaggacctaaaagtaaagctta

SEQ ID NO: 97 - DNA, 23 bp, Cannabis sativa
ctttgggaactgccccaataact

SEQ ID NO: 98 - DNA, 23 bp, Cannabis sativa
tcgaccccactaagttgccctga

SEQ ID NO: 99 - DNA, 23 bp, Cannabis sativa
tgttcacgacacagtcactaatc

SEQ ID NO: 100 - DNA, 23 bp, Cannabis sativa
aatgtgattgtgatcatatgaat

SEQ ID NO: 101 - DNA, 23 bp, Cannabis sativa
tagtgaaagagtaagtgcccaca

SEQ ID NO: 102 - DNA, 23 bp, Cannabis sativa
catttaacaaactcgctattata

SEQ ID NO: 103 - DNA, 23 bp, Cannabis sativa
aaaatctaattttttgtatacta

SEQ ID NO: 104 - DNA, 23 bp, Cannabis sativa
ttaccaggtgcacttttaccttt

SEQ ID NO: 105 - DNA, 23 bp, Cannabis sativa
tttttcataatcgcaaattggcg

SEQ ID NO: 106 - DNA, 23 bp, Cannabis sativa
attatgcttatctaatcgcaaat

SEQ ID NO: 107 - DNA, 23 bp, Cannabis sativa
gatttttacccatttgccctaca

SEQ ID NO: 108 - DNA, 23 bp, Cannabis sativa
atctgatataaattttatcttta

SEQ ID NO: 109 - DNA, 23 bp, Cannabis sativa
tcaaatcactctaaacttactta

SEQ ID NO: 110 - DNA, 23 bp, Cannabis sativa
aatatttgcttcaggttttatcg

SEQ ID NO: 111 - DNA, 23 bp, Cannabis sativa
tggaagagaacggaacattggaa

SEQ ID NO: 112 - DNA, 23 bp, Cannabis sativa
atacttaaaatattgattcatcc

SEQ ID NO: 113 - DNA, 23 bp, Cannabis sativa
tctaacccttgtttcctttctgt

SEQ ID NO: 114 - DNA, 23 bp, Cannabis sativa
cgacatttttctgtggactgtct

SEQ ID NO: 115 - DNA, 23 bp, Cannabis sativa
ttataatgttgggttttatgccc

SEQ ID NO: 116 - DNA, 21 bp, Cannabis sativa
gctctgctacagaggaatcaa

SEQ ID NO: 117 - DNA, 23 bp, Cannabis sativa
cacagacggcgccaaatgttgtc

SEQ ID NO: 118 - DNA, 23 bp, Cannabis sativa
tggctttgtatcgaaattcagtt

SEQ ID NO: 119 - DNA, 23 bp, Cannabis sativa
tataagaggtcggttttgtgtgt

SEQ ID NO: 120 - DNA, 23 bp, Cannabis sativa
ctggttcccccagaacctcttgt

SEQ ID NO: 121 - DNA, 23 bp, Cannabis sativa
cccagtaactatatcaccatcat

SEQ ID NO: 122 - DNA, 23 bp, Cannabis sativa
tggtcagatcaaaacaactttgt

SEQ ID NO: 123 - DNA, 23 bp, Cannabis sativa
caaccaaaacgtttccaaactca

SEQ ID NO: 124 - DNA, 23 bp, Cannabis sativa
caaacttttgtggaaaagatact

SEQ ID NO: 125 - DNA, 23 bp, Cannabis sativa
agataaccgctaactttagggta

SEQ ID NO: 126 - DNA, 23 bp, Cannabis sativa
agtgtcaagtcttctatgtcact

SEQ ID NO: 127 - DNA, 23 bp, Cannabis sativa
gcctcgggtattgtgccgaccga

SEQ ID NO: 128 - DNA, 23 bp, Cannabis sativa
ccatccccgcacgacaccaactg

SEQ ID NO: 129 - DNA, 23 bp, Cannabis sativa
aggcggactactaggagtcaggg

SEQ ID NO: 130 - DNA, 23 bp, Cannabis sativa
agtcgtggctttccattgcagcc

SEQ ID NO: 131 - DNA, 23 bp, Cannabis sativa
tctttaaccataaccactttttt

SEQ ID NO: 132 - DNA, 23 bp, Cannabis sativa
cattgcaaaatcataagacatca

SEQ ID NO: 133 - DNA, 23 bp, Cannabis sativa
atatttttatacatttaactttt

SEQ ID NO: 134 - DNA, 23 bp, Cannabis sativa
gtacttgagtccagagttagtgc

SEQ ID NO: 135 - DNA, 23 bp, Cannabis sativa
acatgttttcctgcggatttcac

SEQ ID NO: 136 - DNA, 23 bp, Cannabis sativa
tcaacattctctttttggtttgt

SEQ ID NO: 137 - DNA, 23 bp, Cannabis sativa
tgtgattgtgtttatcgacgaca

SEQ ID NO: 138 - DNA, 23 bp, Cannabis sativa
aatttcaatgcccctaaccgagc

SEQ ID NO: 139 - DNA, 23 bp, Cannabis sativa
gtagtttgtctcatgttgctcgc

SEQ ID NO: 140 - DNA, 23 bp, Cannabis sativa
ccggtcatactccggctgctagt

SEQ ID NO: 141 - DNA, 23 bp, Cannabis sativa
aatggacctaaataaaatctatc

SEQ ID NO: 142 - DNA, 23 bp, Cannabis sativa
gctccattatataaagctataaa

SEQ ID NO: 143 - DNA, 23 bp, Cannabis sativa
ttacacacaagaaagtatttggg

SEQ ID NO: 144 - DNA, 23 bp, Cannabis sativa
aaaaaaattgaagtatataagaa

SEQ ID NO: 145 - DNA, 23 bp, Cannabis sativa
ccaatactcatcgtataacgccg

SEQ ID NO: 146 - DNA, 23 bp, Cannabis sativa
ccatcccgccacatcctgcttca

SEQ ID NO: 147 - DNA, 23 bp, Cannabis sativa
ctacgctttttgtgtcgtagaaa

SEQ ID NO: 148 - DNA, 23 bp, Cannabis sativa
aacccccataacctcagcaatct

SEQ ID NO: 149 - DNA, 23 bp, Cannabis sativa
ctaggtgcagctcagcctctggc

SEQ ID NO: 150 - DNA, 23 bp, Cannabis sativa
ttattctctcctgaagatgcttc

SEQ ID NO: 151 - DNA, 23 bp, Cannabis sativa
aacctttgcattaagaaggtttt

SEQ ID NO: 152 - DNA, 23 bp, Cannabis sativa
ttcaaaatttcaaagttagaaaa

SEQ ID NO: 153 - DNA, 23 bp, Cannabis sativa
gagtttttgaactcaatcttgaa

SEQ ID NO: 154 - DNA, 23 bp, Cannabis sativa
ttgtaattagtcgctaggcgaca

SEQ ID NO: 155 - DNA, 23 bp, Cannabis sativa
gctttttaatcgacatgatatcg

SEQ ID NO: 156 - DNA, 23 bp, Cannabis sativa
gaatggccgggaatttagaatgt

SEQ ID NO: 157 - DNA, 23 bp, Cannabis sativa
aaataaattaaatttaattttta

SEQ ID NO: 158 - DNA, 23 bp, Cannabis sativa
atgtaatttccattccttaattg

SEQ ID NO: 159 - DNA, 23 bp, Cannabis sativa
gtctcgaagaggacattgttgct

SEQ ID NO: 160 - DNA, 23 bp, Cannabis sativa
ctcaatttcgatttaatttgaat

SEQ ID NO: 161 - DNA, 23 bp, Cannabis sativa
tagcatgctcaccctagtaatca

SEQ ID NO: 162 - DNA, 23 bp, Cannabis sativa
cattttttcatgttttgtgcccg

SEQ ID NO: 163 - DNA, 23 bp, Cannabis sativa
ctcctaatattccctggcgtcca

SEQ ID NO: 164 - DNA, 23 bp, Cannabis sativa
aatttctctctccagaaaaattg

SEQ ID NO: 165 - DNA, 23 bp, Cannabis sativa
tatcgtaatatatattcaattaa

SEQ ID NO: 166 - DNA, 23 bp, Cannabis sativa
actttacgccatataaataactt

SEQ ID NO: 167 - DNA, 23 bp, Cannabis sativa
tatagaggatccttgatcaaatt

SEQ ID NO: 168 - DNA, 23 bp, Cannabis sativa
aaatggcaatagtatttaataat

SEQ ID NO: 169 - DNA, 23 bp, Cannabis sativa
tactaattccagccaacgcctct

SEQ ID NO: 170 - DNA, 23 bp, Cannabis sativa
ataactccttatctgacatggcc

SEQ ID NO: 171 - DNA, 23 bp, Cannabis sativa
atgcttcctataaggcttttctt

SEQ ID NO: 172 - DNA, 23 bp, Cannabis sativa
acaatgtatcaaaattaacaaat

SEQ ID NO: 173 - DNA, 23 bp, Cannabis sativa
tttagtttgttgaatgttatttt

SEQ ID NO: 174 - DNA, 23 bp, Cannabis sativa
caaatacatgccaggtaaaatta

SEQ ID NO: 175 - DNA, 23 bp, Cannabis sativa
caatattctcccaaaattacccc

SEQ ID NO: 176 - DNA, 23 bp, Cannabis sativa
tcactaagcatggacgaaattgt

SEQ ID NO: 177 - DNA, 23 bp, Cannabis sativa
acttaaatatatatttctaatta

SEQ ID NO: 178 - DNA, 23 bp, Cannabis sativa
aaacaacattcatcattttaaat

SEQ ID NO: 179 - DNA, 23 bp, Cannabis sativa
gttatatgtctaaggttatatgt

SEQ ID NO: 180 - DNA, 23 bp, Cannabis sativa
attttcactaaccaggggtgaaa

SEQ ID NO: 181 - DNA, 23 bp, Cannabis sativa
gaatctctactattgcaggacat

SEQ ID NO: 182 - DNA, 23 bp, Cannabis sativa
ctcaacatgtacagaaacaagtt

SEQ ID NO: 183 - DNA, 23 bp, Cannabis sativa
tccttcatttcagttactttcca

SEQ ID NO: 184 - DNA, 23 bp, Cannabis sativa
ggtacctgcagactgaacctgtt

SEQ ID NO: 185 - DNA, 23 bp, Cannabis sativa
cttttaacgacaggataattgta

SEQ ID NO: 186 - DNA, 23 bp, Cannabis sativa
gtaagaaaagagagaaaagatat

SEQ ID NO: 187 - DNA, 23 bp, Cannabis sativa
tgaaccatgcaacattaaatgtt

SEQ ID NO: 188 - DNA, 23 bp, Cannabis sativa
gaaagggaattagtatcaaaaga

SEQ ID NO: 189 - DNA, 23 bp, Cannabis sativa
cacaaacccggactgggaccagg

SEQ ID NO: 190 - DNA, 23 bp, Cannabis sativa
agggtccgggtctggtcgtaatc

SEQ ID NO: 191 - DNA, 23 bp, Cannabis sativa
catcatccccaccacgaacacaa

SEQ ID NO: 192 - DNA, 23 bp, Cannabis sativa
aatagttggcatggaagaaagaa

SEQ ID NO: 193 - DNA, 23 bp, Cannabis sativa
ttgaaaaattaacgtttttgaga

SEQ ID NO: 194 - DNA, 23 bp, Cannabis sativa
cattaattcaggtgcatcaatgt

SEQ ID NO: 195 - DNA, 23 bp, Cannabis sativa
acttaactaaccccagtggagag

SEQ ID NO: 196 - DNA, 23 bp, Cannabis sativa
tgacttgaatatatttttcggta

SEQ ID NO: 197 - DNA, 23 bp, Cannabis sativa
attaaggaaggcagtcttgacat

SEQ ID NO: 198 - DNA, 23 bp, Cannabis sativa
aatataaaacttttaggttataa

SEQ ID NO: 199 - DNA, 23 bp, Cannabis sativa
tgattaatctttgtttattttct

SEQ ID NO: 200 - DNA, 23 bp, Cannabis sativa
taatagatgctacagctggagga

SEQ ID NO: 201 - DNA, 23 bp, Cannabis sativa
aacaatcccaaattgtttgtgag

SEQ ID NO: 202 - DNA, 23 bp, Cannabis sativa
tgaaacagtggcattaactgagg

SEQ ID NO: 203 - DNA, 23 bp, Cannabis sativa
acctaagtgaaatgagatctcca

SEQ ID NO: 204 - DNA, 23 bp, Cannabis sativa
atgaaaaattatcaaattaaagt

SEQ ID NO: 205 - DNA, 23 bp, Cannabis sativa
aaaagctagtggaaaaatgtgcc

SEQ ID NO: 206 - DNA, 23 bp, Cannabis sativa
aagtgctacttttgtggaagggc

SEQ ID NO: 207 - DNA, 23 bp, Cannabis sativa
gggagaagtcagacagacaggtc

SEQ ID NO: 208 - DNA, 23 bp, Cannabis sativa
ttggagtaagtgttatgttcttt

SEQ ID NO: 209 - DNA, 23 bp, Cannabis sativa
tttatttttcgaaaaaataaatt

SEQ ID NO: 210 - DNA, 23 bp, Cannabis sativa
tattttagatactttttaggtta

SEQ ID NO: 211 - DNA, 23 bp, Cannabis sativa
attggtcgttcttttcacattga

SEQ ID NO: 212 - DNA, 23 bp, Cannabis sativa
aattattttttctatatatttta

SEQ ID NO: 213 - DNA, 23 bp, Cannabis sativa
ttattaaataaatagttttgaca

SEQ ID NO: 214 - DNA, 23 bp, Cannabis sativa
atagccttgcctttacctacaca

SEQ ID NO: 215 - DNA, 23 bp, Cannabis sativa
tggtaaaattttcacacaagtca

SEQ ID NO: 216 - DNA, 23 bp, Cannabis sativa
aaaaaattatcgggtctcatata

SEQ ID NO: 217 - DNA, 20 bp, Artificial sequence
  qRT-PCT THCAS forward primer
cagcaattccattccctcat

SEQ ID NO: 218 - DNA, 25 bp, Artificial sequence
  qRT-PCT THCAS reverse primer
ttaggactcgcatgattagtttttc

SEQ ID NO: 219 - DNA, 30 bp, Artificial sequence
  KASP Marker_0 forward primer for G allele
aattagcagtgttaaaatttacaacaccac

SEQ ID NO: 220 - DNA, 32 bp, Artificial sequence
  KASP Marker_0 forward primer for A allele
aaaattagcagtgttaaaatttacaacaccat

SEQ ID NO: 221 - DNA, 30 bp, Artificial sequence
  KASP Marker_0 common reverse primer
atttagctggattgatacaaccatcttcta

SEQ ID NO: 222 - DNA, 22 bp, Artificial sequence
  KASP Marker_Upstream_33 forward primer for T allele
gccatatgctgcatcaaaggca

SEQ ID NO: 223 - DNA, 22 bp, Artificial sequence
  KASP Marker_Upstream_33 forward primer for C allele
gccatatgctgcatcaaaggcg

SEQ ID NO: 224 - DNA, 26 bp, Artificial sequence
  KASP Marker_Upstream_33 common reverse primer
ctatgcagcaaacaccacatacgctt

SEQ ID NO: 225 - DNA, 23 bp, Artificial sequence
  KASP Marker_Downstream_90 forward primer for C allele
cgccattgtttgtgctcgcaaac

SEQ ID NO: 226 - DNA, 23 bp, Artificial sequence
  KASP Marker_Downstream_90 forward primer for T allele
cgccattgtttgtgctcgcaaat

SEQ ID NO: 227 - DNA, 26 bp, Artificial sequence
  KASP Marker_Downstream_90 common reverse primer
cctccgcttctgatcttcatttgcaa

SEQ ID NO: 228 - DNA, 21 bp, Artificial sequence
  KASP Marker_Upstream_121 forward primer for T allele
agttgtacgcgtacgtgccca

SEQ ID NO: 229 - DNA, 20 bp, Artificial sequence
  KASP Marker_Upstream_121 forward primer for G allele
gttgtacgcgtacgtgcccc

SEQ ID NO: 230 - DNA, 27 bp, Artificial sequence
  KASP Marker_Upstream_121 common reverse primer
agacaagctgttggacaataagcacta

SEQ ID NO: 231 - DNA, 1635 bp, Cannabis sativa
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcat

SEQ ID NO: 232 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

(19) United States
(12) Patent Application Publication
(10) Pub. No.: US 2022/0228159 A1
(43) Pub. Date: Jul. 21, 2022

(54) GENETIC LOCUS FOR REGULATING THCAS ACTIVITY IN CANNABIS SATIVA L.

(71) Applicant: Puregene AG, Zeiningen (CH)

(72) Inventors: Gavin M. George, Zurich (CH); Michael E. Ruckle, Zurich (CH); Christelle Cronje, Cape Town (ZA); Yannik Schlup, Zurich (CH)

(21) Appl. No.: 17/150,952

(22) Filed: Jan. 15, 2021

(51) Int. Cl. C12N 15/82; C07K 14/415; C12Q 1/6895

(52) U.S. Cl. CPC C12N 15/8243; C07K 14/415; C12Q 2600/156; C12Q 2600/13; C12Q 1/6895

(57) ABSTRACT

Isolated nucleic acids containing polymorphisms associated with low THCA content have been identified in Cannabis sativa. In one aspect, plants comprising one or more of the isolated nucleic acids are provided. Methods of identifying Cannabis sativa plants that have a low THCA content and plants identified by the methods are also provided. In addition, methods of producing Cannabis sativa plants that comprises a low THCA content and plants produced by this method are provided. Also disclosed are methods of marker assisted selection and marker assisted breeding to obtain plants having a low THCA content.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### SEQUENCE LISTING

The instant application contains a Sequence Listing which has been submitted via EFS-Web and is hereby incorporated by reference in its entirety. Said ASCII copy, created on Jan. 15, 2021, is named PCR006ASEQLIST.txt, and is 49,775 bytes in size.

### BACKGROUND

### Field

The present disclosure relates to nucleic acids comprising SNPs that are associated with a low-THCA trait in Cannabis plants, and to Cannabis plants comprising the nucleic acids. The disclosure also relates to methods of identifying Cannabis plants that have a low-THCA trait and plants with reduced levels of THCA identified by the methods. The disclosure further relates to marker assisted selection and marker assisted breeding methods for obtaining plants having a low-THCA trait, as well as to methods of producing Cannabis plants with reduced levels of THCA and plants produced by these methods.

### Background

Modern Cannabis is derived from the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, called Hemp and HRT (high-resin-type) Cannabis, respectively, which are used for different purposes. Hemp is primarily used for industrial purposes, for example in feed, food, seed, fiber, and oil production. Conversely, high-resin-type (HRT) Cannabis is largely cultivated and bred for high concentrations of the pharmacological constituents, cannabinoids, derived from resin in the trichomes. However, there is recent interest from industrial producers in valuable, novel varieties based on the convergence of these two types.

Cannabis is the only species in the plant kingdom to produce phytocannabinoids. Phytocannabinoids are a class of terpenoid acting as antagonists and agonists of mammalian endocannabinoid receptors. The pharmacological action is derived from this ability of phytocannabinoids to disrupt or mimic endocannabinoids. Due to its psychoactive properties, one cannabinoid, delta-9-tetrahydrocannabinol (THC), the decarboxylation product of the plant-produced THCA, has received much attention in illegal or unregulated breeding programs, with modern HRT varieties having THC concentrations of 0.5% to 30%.

In the US Cannabis plants and derivatives that contain no more than 0.3 percent THC on a dry weight basis are no longer controlled substances under federal law (Agriculture Improvement Act of 2018, Pub. L. 115-334). However, several varieties of the industrial hemp type, which are grown for fiber and seed production, have been shown to accumulate more than 0.3% THC. This transfers the burden of complicated testing to farmers to ensure that their crops are legal throughout the life cycle to obtain an unrelated agricultural product. Thus, eliminating THC can provide added utility to these agricultural crops.

In addition, while being psychoactive, THC is not always the focus of pharmaceutical applications. Cannabis also produces over 100 other cannabinoids, most notably cannabidiol (CBD), cannabigerol (CBG), and cannabichromene (CBC). Indeed, a full-spectrum Cannabis extract containing THC can have several undesirable side effects like dry mouth, anxiety, psychotic events, compromised cognition and motor function, paranoia, and erectile dysfunction. As such, irrespective on any legality considerations, industrial production can also benefit from the elimination/reduction of THCA from Cannabis.

CBGA is the precursor molecule of THCA, synthesized by THCA synthase (THCAS). CBDA is also synthesized from the CBGA precursor by a similar, but functionally distinct synthase enzyme.

### SUMMARY

In one aspect, the present disclosure relates to nucleic acids having a nucleotide sequence comprising single nucleotide polymorphisms (SNPs) that are associated with a low tetrahydrocannabinolic acid (THCA) trait in Cannabis plants (low-THCA trait), and to Cannabis plants, seeds or plant parts comprising the nucleic acids. Also provided are methods of identifying Cannabis sativa plants that have a low-THCA trait and plants with reduced levels of THCA identified by the methods. Marker assisted selection and marker assisted breeding methods for obtaining plants having a low-THCA trait, as well as to methods of producing Cannabis plants with reduced levels of THCA and plants produced by these methods are encompassed, as discussed in more detail below.

In some embodiments, isolated nucleic acids are provided comprising a polymorphism associated with low tetrahydrocannabinolic acid (THCA) content in Cannabis sativa. In some embodiments the polymorphism is a single nucleotide polymorphism (SNP). In some embodiments the single nucleotide polymorphism is one of the SNPs described in SEQ ID NOs: 3-216. In some embodiments the isolated nucleic acid comprises a sequence that is fully complementary to an isolated nucleic acid comprising one of the SNPs described in SEQ ID NOs: 3-216. In some embodiments the isolated nucleic acid is selected from the group consisting of SEQ ID NOs: 3-216. In some embodiments the isolated nucleic acid comprises a sequence that is fully complementary to an isolated nucleic acid selected from the group consisting of SEQ ID NOs: 3-216.

In some embodiments isolated nucleic acids are provided comprising a single nucleotide polymorphism associated with low THCA content in Cannabis sativa, wherein the isolated nucleic acid is selected from the group consisting of a) SEQ ID NO: 3; b) a nucleotide sequence that is 90% identical to SEQ ID NO: 1 and retains the G1064A single nucleotide polymorphism; and c) a sequence that is fully complementary to the sequence of a) or b).

In some embodiments the isolated nucleic acid comprises a nucleotide sequence that is at least 90% identical to SEQ ID NO: 1 and retains a polymorphism from any of SEQ ID NOs: 3-216. In some embodiments the isolated nucleic acid comprises a sequence that is fully complementary to an isolated nucleic acid that is at least 90% identical to SEQ ID NO: 1 and retains a polymorphism from any of SEQ ID NOs: 3-216.

In some embodiments, isolated nucleic acids are provided that have a nucleotide sequence having a single nucleotide polymorphism associated with low tetrahydrocannabinolic acid (THCA) content in Cannabis sativa, wherein the isolated nucleic acid is selected from the group consisting of a) SEQ ID NO: 3; b) a nucleotide sequence that is 90% identical to SEQ ID NO: 1 and retains the G1064A single nucleotide polymorphism; and c) a sequence that is fully complementary to the sequence of a) or b).

In some embodiments an isolated nucleic acid having 90% sequence identity to SEQ ID NO: 1 are provided, wherein the nucleic acid comprises the single nucleotide polymorphism G1064A or C998G. In some embodiments the nucleic acid comprises the SNP G1064A. In some embodiments the isolated nucleic acid encodes a mutant THCAS enzyme having decreased activity compared to a reference THCAS enzyme that does not comprise the SNP. In some embodiments the isolated nucleic acid encodes a mutant THCAS enzyme having decreased activity compared to a THCAS enzyme having the amino acid sequence of SEQ ID NO: 232. In some embodiments the isolated nucleic acid comprises the nucleic acid sequence of SEQ ID NO: 1.

In some embodiments a plant, seed or plant part of Cannabis sativa L. is provided, comprising one or more of the isolated nucleic acids.

In some embodiments, a mutant tetrahydrocannabinolic acid synthase (THCAS) enzyme is provided, having the amino acid sequence set forth in SEQ ID NO:2. In some embodiments a mutant tetrahydrocannabinolic acid synthase (THCAS) enzyme is provided having the amino acid sequence 90% identical, 95% identical, 97% identical, 98% identical, 99% identical, or 100% identical to the sequence set forth in SEQ ID NO:2. In some embodiments the mutant THCAS enzyme has decreased activity compared to a reference THCAS enzyme having the amino acid sequence of SEQ ID NO:232.

In some embodiments a Cannabis sativa plant is provided comprising a mutant THCAS gene with 90% sequence identity to SEQ ID NO:1, wherein the nucleic acid comprises the single nucleotide polymorphism (SNP) G1064A or C998G. In some embodiments the Cannabis sativa plant has a concentration of less than 0.1% THCA in the dry weight (DW) of the mature inflorescence. In some embodiments plant extracts obtained from such plants are provided. In some embodiments the plant extract may be characterized by the unique Cannabinoid and Terpene profile as shown in Table 1. In some embodiments the plant extract may have a THCA concentration of less than 0.1%. In some embodiments the plant extract contains >0.1% THCA, <0.1% CBDA and <0.1% CBCA. In some embodiments the plant extract contains >0.1% THCA and >1% CBDA and/or >1% CBCA.

In some embodiments, methods for identifying a Cannabis sativa plant that comprises a low THCA content are provided. The methods may comprise detecting at least one polymorphism in the grTHC1.1 genomic region. In some embodiments the at least one polymorphism may comprise at least one of the polymorphisms of SEQ ID NOs: 3-216. In some embodiments the polymorphism is a single nucleotide polymorphism (SNP) selected from the group consisting of M0, MU35, MD90, or MU123. These SNPs are descried in SEQ ID NOs:3-6 respectively. In some embodiments the methods comprise detecting a haplotype comprising the G1064A SNP from SEQ ID NO: 3 and one or more additional SNPs selected from the marker loci of SEQ ID NOs: 4-216. In some embodiments Cannabis sativa plants are provided that have been identified by the methods. In some embodiments seed from the Cannabis sativa plants is provided. In some embodiments plant extracts obtained from the identified plants are provided. In some embodiments plant extracts obtained from such plants are provided. In some embodiments the plant extract may be characterized by the unique Cannabinoid and Terpene profile as shown in Table 1. In some embodiments the plant extract may have a THCA concentration of less than 0.1%. In some embodiments the plant extract contains >0.1% THCA, <0.1% CBDA and <0.1% CBCA. In some embodiments the plant extract contains >0.1% THCA and >1% CBDA and/or >1% CBCA.

In some embodiments methods for identifying a Cannabis sativa plant that comprises a low THCA content are provided comprising detecting at least one allele of a marker locus, wherein the marker locus is a sequence comprising a single nucleotide polymorphism (SNP) located within a chromosomal interval comprising and flanked by SEQ ID NO: 6 and SEQ ID NO: 5. In some embodiments the SNP is associated with a low THCA content. In some embodiments the marker locus is selected from the group consisting of any one of SEQ ID NOs:3-216. In some embodiments the marker locus is selected from the group consisting of any one of SEQ ID NOs:3-6. In some embodiments the methods comprise detecting a haplotype comprising a plurality of the marker alleles. In some embodiments the SNP is G1064A or is in linkage disequilibrium with G1064A. In some embodiments Cannabis sativa plants are provided that have been identified by the methods. In some embodiments seed from the Cannabis sativa plants is provided. In some embodiments plant extracts obtained from the identified plants are provided. In some embodiments plant extracts obtained from such plants are provided. In some embodiments the plant extract may be characterized by the unique Cannabinoid and Terpene profile as shown in Table 1. In some embodiments the plant extract may have a THCA concentration of less than 0.1%. In some embodiments the plant extract contains >0.1% THCA, <0.1% CBDA and <0.1% CBCA. In some embodiments the plant extract contains >0.1% THCA and >1% CBDA and/or >1% CBCA.

In some embodiments methods of producing Cannabis sativa plants are provided. In some embodiments, the methods comprise introducing one or more SNPs selected from the SNPs of any of SEQ ID NOs: 3-216 into a Cannabis sativa plant. In some embodiments the methods comprise introducing one or more single nucleotide polymorphisms (SNPs) selected from the SNPs shown in Table 2 into a Cannabis sativa plant, wherein the SNP is associated with low THCA content. In some embodiments the THCA content in dry weight (DW) of the mature inflorescence of the Cannabis sativa plant in which the one or more SNPs have been introduced is reduced relative to a Cannabis plant in which the one or more SNPs have not been introduced. In some embodiments introducing the one or more SNPs comprises crossing a donor parent plant in which the one or more SNPs is present with a recipient parent plant in which the one or more SNPs is not present. In some embodiments introducing the one or more SNPs comprises genetically modifying the Cannabis sativa plant by mutagenesis and/or gene editing. In some embodiments the SNP G1064A is introduced into a Cannabis sativa plant. In some embodiments Cannabis sativa plants produced by the methods are provided. In some embodiments seed from the Cannabis sativa plants is provided. In some embodiments plant extracts obtained from the plants produced by the methods are provided. In some embodiments plant extracts obtained from such plants are provided. In some embodiments the plant extract may be characterized by the unique Cannabinoid and Terpene profile as shown in Table 1. In some embodiments the plant extract may have a THCA concentration of less than 0.1%. In some embodiments the plant extract contains >0.1% THCA, <0.1% CBDA and <0.1% CBCA. In some embodiments the plant extract contains >0.1% THCA and >1% CBDA and/or >1% CBCA.

In some embodiments, methods of marker assisted selection of Cannabis sativa plants are provided. A population of Cannabis sativa plants can be screened for plants having at least one allele of a marker locus. In some embodiments the marker locus comprises a SNP selected from the SNPs of SEQ ID NO: 3-216. In some embodiments the SNP is associated with low THCA content. In some embodiments the marker locus is a sequence comprising a single nucleotide polymorphism (SNP) selected from the group consisting of any one of SEQ ID NOs: 3-216, wherein the SNP is associated with low THCA content. In some embodiments the marker locus is selected from the group consisting of any one of SEQ ID NOs: 3-6. In some embodiments the marker locus is the single nucleotide polymorphism (SNP) G1064A is in linkage disequilibrium with the G1064A SNP. Plants are selected comprising the at least one allele of the marker locus.

In some embodiments methods of marker assisted breeding are provided. In some embodiments the methods comprise providing a Cannabis sativa donor parent plant having at least one allele of a marker locus associated with low THCA content. In some embodiments the marker locus is identified by marker assisted selection. For example, a population of Cannabis sativa plants can be screened for plants having at least one allele of a marker locus, wherein the marker locus is the single nucleotide polymorphism (SNP) G1064A in the nucleic acid of SEQ ID NO: 3 or is in linkage disequilibrium with the G1064A SNP and selecting a plant comprising the at least one allele to serve as a donor plant. In some embodiments the marker locus is selected from the group consisting of any one of SEQ ID NOs: 3-6. The donor plant is crossed with a recipient parent plant and the progeny are evaluated for the presence of the at least one allele. Progeny having the at least one allele may be selected. Progeny Cannabis plants resulting from the marker assisted breeding methods are also provided. In some embodiments seed from the Cannabis sativa plants is provided. In some embodiments plant extracts obtained from plants produced by the marker assisted breeding are provided. In some embodiments plant extracts obtained from such plants are provided. In some embodiments the plant extract may be characterized by the unique Cannabinoid and Terpene profile as shown in Table 1. In some embodiments the plant extract may have a THCA concentration of less than 0.1%. In some embodiments the plant extract contains >0.1% THCA, <0.1% CBDA and <0.1% CBCA. In some embodiments the plant extract contains >0.1% THCA and >1% CBDA and/or >1% CBCA.

In some embodiments the plant, Cannabis sativa plants, plant parts, seeds and/or plant extracts provided may be used therapeutically, for example in a method of treatment of cancer, pain, infection, inflammation, Glaucoma, and/or cardiovascular disease. In some embodiments methods of treatment of cancer, pain, infection, inflammation, Glaucoma, and/or cardiovascular disease comprise administering said Cannabis sativa plants, plant parts and/or plant extract to a subject in need thereof.

According to a further aspect the plants, seeds, plant parts of Cannabis sativa L. as described herein, or the plant extract as described herein, may be used non-medically, for example as a smokeable and/or tobacco replacement product.

Also provided for are products comprising Cannabis sativa plants as described herein, parts thereof and/or extracts thereof, and methods of preparing such products. For example, said Cannabis sativa plant, parts thereof and/or extracts thereof may be used to prepare cigarettes or micronic compositions.

### BRIEF DESCRIPTION OF THE DRAWINGS

Non-limiting embodiments of the invention will now be described by way of example only and with reference to the following figures:

FIG. 1: UPLC chromatograph of a mixture of cannabinoid standards (top panel) and an extract of PG_1_19_0125_0002 (bottom panel).

FIG. 2: An alignment of a region of the ARG3 reference genome which is homologous to the genomic region grTHC1.1, to the publicly available cs10 Cannabis genome. The light grey blocks of ARG3 align to the light grey blocks of cs10 as shown by the dark grey connecting lines. Dark grey blocks align to other chromosomes of cs10 and are not shown. The light grey blocks on the cs10 chromosome 9 are enlarged 4 fold for clarity.

FIG. 3: A multiple sequence alignment of several plants' homologous nucleotide sequences of the THCAS. The analysis includes the THCAS from PG_1_19_0125_0002 and all 57 THCAS gene sequences available from the NCBI database. A cut-off of 98% similarity was applied. The polymorphism at position 1064 is unique to the THCAS from PG_1_19_0125_0002 .

FIG. 4: Nucleotide sequence of the non-functional THCAS in PG_1_19_0125_0002 (SEQ ID NO:1) aligned with the ARG3 reference genome, showing the identified SNP at position 1064 of the gene, which results in an amino acid change from serine to asparagine.

FIG. 5: Amino acid sequence of amino acids 304-367 of the THCAS enzyme in PG_1_19_0125_0002 (SEQ ID NO:2) aligned with the amino acid sequence of amino acids 304-367 of the THCAS enzyme of ARG3 (SEQ ID NO:232) showing the amino acid changes at amino acid positions 333 (P333R) and 355 (S355R).

FIG. 6: Relative expression of THCAS gene in PG_1_19_0125_0002 and various plants that produce >0.1% THCA in the dry mass of the mature inflorescence.

FIG. 7: Distribution of chemotypes identified in 95 plants in an F2 population showing the typical, 1:3, Mendelian segregation of the recessive low-THCA trait. Cannabinoids in the plants were measured using chromatography methods described in the art.

FIG. 8: Using a KASP molecular marker (KASP03) which detects the SNP at position 1064 of SEQ ID NO:1, the 96 plants of the segregating F2 population were genotyped. The genotypic groups are indicated as homozygous (Alt) or the “low-THCA allele”, homozygous (Ref) or the “high-THCA allele”, or heterozygous. The marker perfectly describes the cannabinoid phenotype measured in the plants.

FIG. 9: A schematic representation of the 2.5 Mb genomic region of PG_1_19_0125_0002 termed grTHC1.1 including the relative positions of the molecular markers KASP14, KASP06, KASP03, and KASP09, which detect the polymorphisms “Marker_Upstream_123”, “Marker_Upstream_35”, “Marker_0”, and “Marker_Downstream_90” respectively. The estimated recombination frequencies between each polymorphism is shown.

### SEQUENCE LISTING

The nucleic acid and amino acid sequences listed herein and in any accompanying sequence listing are shown using standard letter abbreviations for nucleotide bases, and the standard one or three letter abbreviations for amino acids. It will be understood by those of skill in the art that only one strand of each nucleic acid sequence is shown, but that the complementary strand is included by any reference to the displayed strand.

### DETAILED DESCRIPTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

Molecular analytic tools can be used to breed Cannabis varieties, including for commercial and research use. Genomic regions controlling the production of cannabinoids, such as the production of THCA can be identified using these tools. Genetic or molecular markers to these regions can be used in Cannabis breeding to identify plants with a desired phenotype, such as low THCA content due to the disruption of THCA production. Methods and compositions for providing a plant with a desirable cannabinoid profile are provided, along with related compositions and plants.

Cannabis varieties have been identified and described herein that have extremely low levels of THCA, while continuing to produce other useful cannabinoids, including CBGA, CBDA and CBCA. Polymorphisms, including a number of single nucleotide polymorphisms (SNPs) associated with the low-THCA trait were identified in Cannabis sativa. Table 2 herein provides a number of polymorphisms which define the haplotype of the genomic region associated with the low-THCA trait, termed grTHC1.1. In some embodiments one or more of the identified SNPs can be used to incorporate the low-THCA trait from a donor plant (containing the low-THCA trait) into a recipient plant, preferably with other desirable traits, thereby creating a new variety with both the desirable traits of the recipient plant, and the low-THCA phenotype of the donor plant. For example, the incorporation of the low-THCA phenotype may be performed by crossing a donor plant to a recipient to produce plants containing a haploid genome from both parents. Recombination of these genomes provides F1 progeny where each haploid complement of chromosomes, of the diploid genome, is comprised of genetic material from both parents.

In some embodiments, methods of identifying a specific THCAS allele containing one or more of the identified polymorphisms are provided. This THCAS allele forms part of a larger genomic region, termed grTHC1.1 that is characterized by a haplotype comprising of a series of homozygous polymorphisms in linkage disequilibrium. This genomic region is frequently inherited as a unit due to the limited frequency of recombination within the region. Preferably the polymorphisms are selected from Table 2 herein. KASP molecular markers have been designed that can be used to detect the presence of the polymorphisms. These markers have been shown to accurately detect the presence of the genomic region and the specific THCAS allele and whether it is either homozygous or heterozygous in a plant. The identified SNPs and the associated molecular markers can be used in a Cannabis breeding program. The molecular markers can predict the low-THCA chemotype of plants in a breeding population and can be used to produce Cannabis plants in which THCA is reduced or eliminated. For example, THCA levels can be reduced in a progeny plant relative to a recipient parent plant by crossing the recipient parent plant with a donor plant. In particular, the THCA levels may be reduced such that the progeny contains less than 10%, less than 5%, less than 1%, less than 0.5%, or less than 0.1% THCA than that found in a recipient parent plant.

As used herein, reference to a plant or a variety with “low-THC”, “low-THCA”, or “THC-free” refers to a plant or a variety that has a THCA content in the dry weight (DW) of the mature inflorescence below 0.1% THCA before decarboxylation.

As used herein, reference to a plant or a variety with “high-THC” or “high-THCA” refers to plants that produce more than 0.1% THCA in the DW of the mature inflorescence before decarboxylation.

As used herein, the term “low-THCA polymorphisms” refers to the polymorphism denoted as “Marker_0” that best predicts the presence or absence of THCA in a plant as well as Marker_Upstream_1 to 123, and Marker_Downstream_1 to 90 as described in Table 2.

As used herein, the term “low-THCA haplotype” refers to the nucleotide sequence within and around the genomic region, which is referred to herein as “grTHC1.1” comprising of at least two or more of the low-THCA-associated polymorphisms.

As used herein, the term “donor parent plant” refers to a plant, plant part, seed, gamete, or plant cell that is either homozygous or heterozygous for the low-THCA trait or which contains one or more of the low-THCA polymorphisms or the low-THCA haplotype disclosed herein.

As used herein, the term “recipient parent plant” refers to a plant that is heterozygous or homozygous for the high-THCA trait or which is not homozygous for the low-THCA polymorphism Marker_0 or parts of the low-THCA haplotype, disclosed herein, that would result in the low-THCA phenotype.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

“low-THCA allele” is the allele at a particular locus that confers, or contributes to, low-THCA phenotype. A “low-THC marker allele” is a marker allele that segregates with the allele that confers, or contributes to, low-THCA phenotype, or alternatively, is an allele that allows the identification of plants with low-THCA phenotype that can be included in a breeding program (“marker assisted breeding” or “marker assisted selection”).

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium are considered linked.

The term “nucleic acid” encompasses both ribonucelotides (RNA) and deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

The term “isolated”, as used herein means having been removed from its natural environment.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide or nucleic acid sequence that differs from a reference sequence only by one or more conservative substitutions, or by one or more non-conservative substitutions, deletions, or insertions located at positions of the sequence that do not destroy or substantially reduce the antigenicity of the expressed fusion protein or of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein. In one embodiment of the current invention, the nucleic acid sequence is the one provided in SEQ ID NO: 3, which forms part of a THCAS gene (SEQ ID NO: 1) containing a single nucleotide polymorphisms (Marker_0) at position 1064 presenting as an adenine rather that a guanine (G1064A) when compared to SEQ ID NO: 231. In some embodiments the nucleic acid sequences are those substantially homologous to SEQ ID NO: 1 or SEQ ID NO: 3 but retaining the specific Marker_0 polymorphism or the complementary polymorphism G1046A of SEQ ID NO: 1.

Alternatively, or additionally, two nucleic acid sequences may be “substantially identical” or “substantially homologous” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

### Methods of Identifying a Genetic Locus or Haplotype Responsible for Low-THCA Phenotype and Molecular Markers Therefor

In some embodiments, methods are provided for identifying a genomic region or haplotype responsible for reduced THCA production in a Cannabis plant, such as a genomic region or haplotype in Cannabis sativa that prevents or limits THCA accumulation when present in a homozygous state. In some embodiments, the methods may comprise the steps of:

1. Providing a population of Cannabis plants created by crossing one or more Cannabis plants. In some embodiments at least one of the parent plants displays a THC content of <0.1% DW or contains the genomic region or haplotype controlling the low-THC trait in one or more of the gametes that result in the population. In some embodiments the one or more Cannabis plants, or parts thereof, contain a sequence of nucleic acids that result in a THC content that is <0.3%, <0.2% or <0.1% DW. In some embodiments the crossing of parent plants allows for mutations that occur as part of a breeding process to be fixed in the genome of one or more plants of the resultant population.

2. In some embodiments the plants of the population are analyzed for the THCA content and preferably one or more of CBGA content, CBCA content, CBDA content, and/or other non-THCA cannabinoids. In some embodiments, the quantitative measurements of the cannabinoid content is performed on the mature flower.

3. Using the phenotypic data from the population to identify plants with desirable chemotypes. In some embodiments the desirable chemotype may be one or more chemotypes selected from the group consisting of: low THC concentration of <0.3%, <0.2% or <0.1% DW, CBGA concentration of >2% DW, CBCA concentration of >0.2% DW, CBDA concentration of >0.2% DW, and detectable levels of non-THCA cannabinoids.

4. Sequencing the genomes of one or more of the plants identified as having desirable chemotypes, alternatively sequencing an amplicon of a relevant region obtained by polymerase chain reaction (PCR);

5. Comparing the genome sequences of the plants identified as having desirable chemotypes with the genome sequences of other Cannabis plants that have a high-THC phenotype;

6. Analyzing the sequences to identify haplotypes of one or more polymorphisms, for example, SNPs, sequence insertions, sequence deletions, or other sequence polymorphisms, that are associated with the low-THCA trait, wherein the polymorphisms may comprise unique polymorphisms.

7. Optionally, correlating the identified haplotypes with a reduced THC content in varieties that contain high levels of other cannabinoids.

The identified polymorphisms can be either causative or linked to a causative polymorphism. For the purposes of identifying the genomic regions or haplotypes associated with a trait, causative and non-causative polymorphisms become more similarly useful the greater the linkage disequilibrium between them. These genomic regions or haplotypes contain polymorphisms that are strongly linked with a low chance of recombination between them and can effectively be used to determine the presence or absence of the trait in the breeding population using molecular markers which may be specific PCR primers, labelled probes, or any other tool of molecular biology that can differentiate polymorphisms at a locus.

In some embodiments, methods are provided for marker assisted breeding (MAB) or marker assisted selection (MAS) of plants having a low-THCA trait. The methods may comprise the steps of:

1. Providing one or more Cannabis plants, Cannabis plants from a breeding program, or a Cannabis germplasm collection that contains a high level of genetic diversity. In some embodiments the collection comprises two or more distinct plants. In some embodiments the collection comprises >100 visually distinct plants. In some embodiments the plants may have diverse cannabinoid and/or terpene profiles.

2. Identifying Cannabis plants from the provided plants that contain a low-THC phenotype but that do produce other cannabinoids that accumulate to greater than 2% of the DW in the mature inflorescence.

3. Identifying a genomic region or haplotype responsible for low-THCA trait in a Cannabis plant obtained from the provided plants;

4. Identifying one or more unique and rare SNPs within the identified genomic region that make up the haplotype;

5. Designing molecular markers to detect the presence or absence of the SNPs;

6. Identifying one, or more, Cannabis varieties containing the SNPs and using it to create a breeding population containing the low-THCA trait.

7. Using molecular markers to identify plants within the breeding population containing or lacking the polymorphisms and then select plants from this and subsequent generation populations containing the trait or traits linked to the previously identified polymorphisms.

### Polymorphisms Associated with the Low-THCA Trait

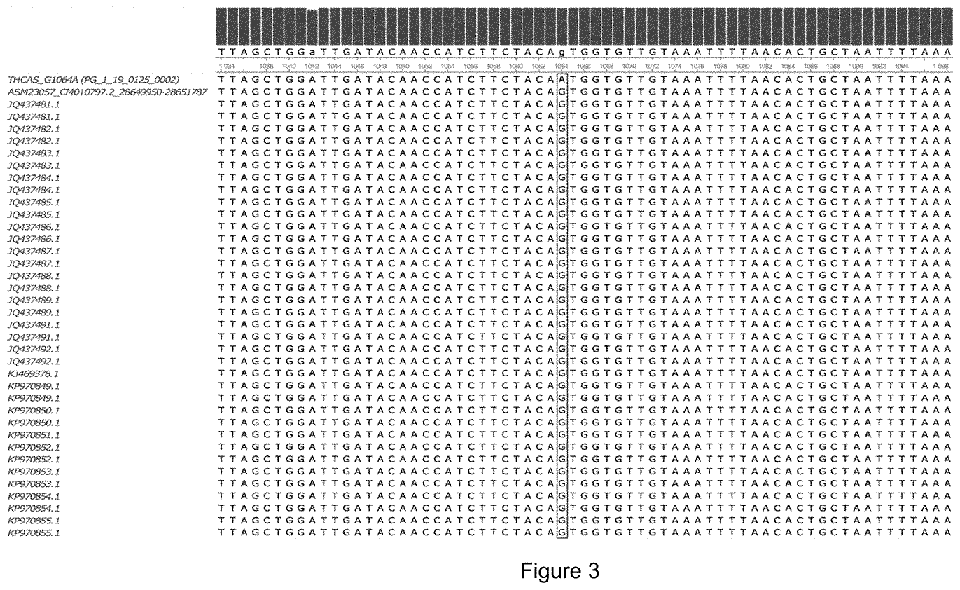

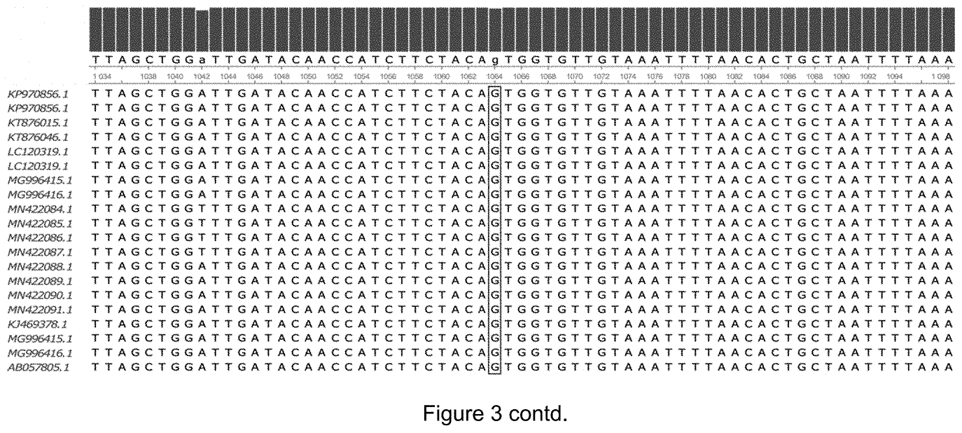

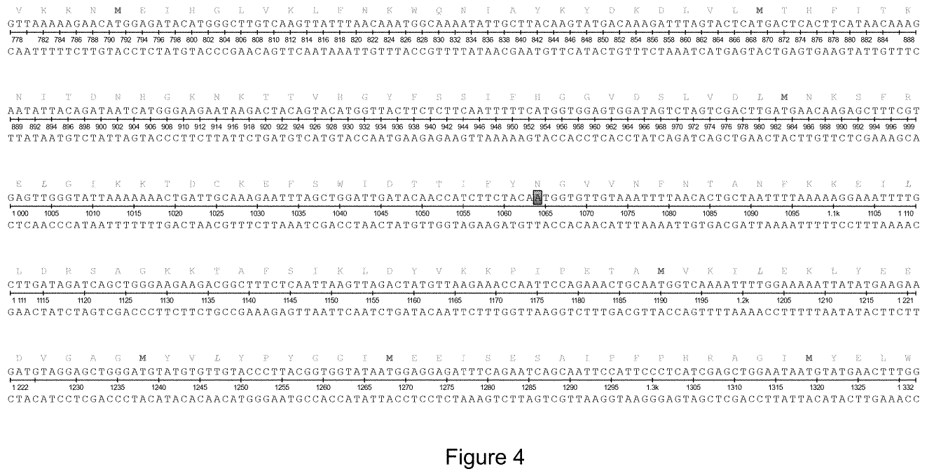

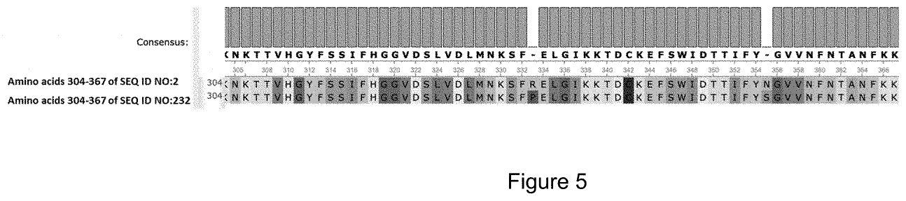

With reference to the THCAS gene (THCAS_{G1064A}) having the sequence disclosed in FIG. 3 (SEQ ID NO:1), an allele containing a SNP at position 1064 when compared with the reference ARG3 sequence of SEQ ID NO:231 has been identified. This SNP is referred to herein as Marker_0, and comprises an adenine at position 1064 (SEQ ID NO:1) rather than a guanine at position 1064 (SEQ ID NO:231), resulting in an amino acid change from a serine (amino acid 355 of SEQ ID NO:232) to an asparagine at position 355 as shown in SEQ ID NO:2 (FIG. 4). The THCAS_{G1064A} A polymorphism (designated herein as Marker_0) in the THCAS_{G1064A} gene results in an amino acid change in the translated protein and therefore likely affects the activity of the enzyme. A further SNP in the THCAS gene was identified at position 998 of SEQ ID NO:1, which comprises a guanine at position 998 of the gene with reference to SEQ ID NO:1 instead of a cytosine in the corresponding position of the ARG3 THCAS gene (SEQ ID NO:231), and results in an amino acid change from a proline to arginine at amino acid position 333 of SEQ ID NO:2 when compared with the reference sequence of SEQ ID NO:232 (FIG. 5). In addition, the inventors of the present invention have identified additional SNPs within the same genetic region that can be the targets of molecular markers and that accurately predict the low-THCA trait (see Table 2).

In some embodiments, a single linked polymorphism is sufficient to validate the inheritance of a genomic region in a breeding population and the trait encoded by this region, as shown in the examples below. By way of illustration, plants in a population were found to consistently display the low-THCA trait when the Marker_0 SNP is present in a homozygous state.

In some embodiments one or more SNPs disclosed in Table 2 are used to identify plants with the low THCA trait.

The disclosed SNPs can be detected using any number of techniques including direct DNA or genome sequencing, restriction enzyme digestion of PCR products, or by using molecular markers.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype. Alternatively, the marker may introduce a restriction enzyme recognition site, or result in the removal of a restriction enzyme recognition site. Plants can be screened for a particular trait based on the detection of one or more markers confirming the presence of the polymorphism. Markers detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system and may be PCR primers, such as those described in the examples below. For example, PCR primers may be designed that consist of a reverse primer and two forward primers that are homologous to the part of the genome that contains a SNP but differ in the 3′ nucleotide such that the one primer will preferentially bind to sequences containing the SNP and the other will bind to sequences lacking it. The three primers are used in single PCR reactions where each reaction contains DNA from a plant as a template. Fluorophores linked to the forward primers provide, after thermocycling, a different relative fluorescent signal for homozygous and heterozygous alleles containing the SNP and for those lacking it, respectively. For example, providing DNA extracted from individual plants of a population into individual PCR reactions containing the three primers in Table 2 of the examples below allows for the identification of the state of the THCAS_{G1064A} SNP in each of the plants.

In some embodiments, allele-specific primers may each harbor a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals, and is evaluated with commercial software.

If the genotype at a given SNP is homozygous, one of the two possible fluorescent signals will be generated. If the genotype is heterozygous, a mixed fluorescent signal will be generated. By way of example, genomic DNA extracted from Cannabis leaf tissue at seedling stage can be used as a template for PCR amplifications with reaction mixtures containing the three primers. Final fluorescent signals can be detected by a thermocycler and analyzed using standard software for this purpose, which discriminates between individuals that are heterozygotes or homozygotes for either allele.

In some embodiments, molecular markers to one, two or more of the SNPs in the haplotype can be used to identify the presence of the SNPs and by association, the low-THCA phenotype.

Further, the genomic region may include a number of individual SNPs in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the THCA-related phenotype of the offspring.

### Larger Genomic Region Responsible for the Low-THCA Phenotype

The Marker_0 polymorphism described above is a single example of several polymorphisms that exist in a larger genomic region which is shown herein to control and/or predict the low-THC phenotype. As the Marker_0 polymorphism exists in a THCAS gene it is plausible that this SNP is partially or completely responsible for the low-THC phenotype, however, it may be that any other features of the genomic region either contribute to the phenotype or are entirely responsible. Moreover, as the genomic region is relatively small, it is generally inherited as a unit due to the low rate of recombination in this small region of the genome during sexual reproduction. Here the Marker_0 SNP-containing region is described by defining a series of polymorphisms contained within the region and by comparing it to similar regions that exist in other Cannabis plants.

A plant that comprises a genomic region, grTHC1.1, responsible for the low-THC phenotype has been identified and is designated PG_1_19_0125_0002. This variety displays a phenotype of <0.1% THCA in the dry weight of the mature inflorescence.

A genomic region, designated as grTHC1.1, associated with the low-THC trait, within PG_1_19_0125_0002 was identified and found to contain all of the polymorphisms listed in Table 2. In general, the closer two polymorphisms are on a genome the lower the chances of recombination during sexual reproduction. Therefore, in some embodiments molecular markers designed to detect any one or more of the polymorphisms described in Table 2 of the examples below may be used to track the genomic region in a breeding program. Thus, in some embodiments molecular markers to any one or more of the polymorphisms described in Table 2 (SEQ ID NO: 3 to 216) are used to identify the presence of the polymorphisms and the trait, or the potential to contain the trait in a subsequent generation.

### Construction of Breeding Populations

Breeding populations are the offspring of sexual reproduction events between two or more parents. The parent plants (F0) are crossed to create an F1 population each containing a chromosomal complement of each parent. In a subsequent cross (F2) recombination has occurred and allows for mostly independent segregation of traits in the offspring and importantly the reconstitution of recessive phenotypes that existed in only one of the parental lines.

In some embodiments a low-THC phenotype plant, such as an offspring of PG_1_19_0125_0002, or a plant derived from PG_1_19_0125_0002 can be used as a donor parent plant providing the low-THC trait to a breeding population. In some embodiments the low-THC phenotype plant is one comprising any one or more of the low-THCA polymorphisms in Table 2, such as the Marker_0 SNP in THCAS_{G1064A}.

In one embodiment the one or more low-THCA polymorphisms, such as those described in Table 2, may be used to identify a donor parent plant (F0) with the low-THC trait. In some embodiments a high-THC recipient parent plant is crossed with the donor parent plant through traditional breeding methods in order to transfer the genomic region through recombination into several offspring.

In some embodiments, the donor parent plant is heterozygous for the haplotype controlling the low-THCA trait and so progeny of the cross (F1) are provided and screened with molecular markers such as those disclosed herein, to identify those carrying the haplotype controlling the low-THC trait.

According to some embodiments, any polymorphism in linkage disequilibrium with a low-THCA trait can be used to determine the presence of the haplotype in a breeding population of plants, as long as the polymorphism is unique to the low-THC trait in the donor parent plant when compared to the recipient parent plant.

In some embodiments of the invention, the donor parent plant is a plant that has been genetically modified to include any one or more of the low-THCA polymorphisms in Table 2. In some embodiments the donor plant comprises the Marker_0 SNP. The donor parent plant may also be obtained by crossing and selection for plants that display the low-THC-trait or that contain one or more of the low-THCA polymorphisms.

In some embodiments, donor parent plants, as described above, are used as one of two parents to create breeding populations (F1) through sexual reproduction. Methods for reproduction that are known in the art may be used. The donor parent plant provides the trait of interest to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a low-THCA allele or low-THCA haplotype containing the Marker_0 SNP.

In some embodiments, the presence of the low-THCA allele or low-THCA haplotype in plants to be used in the F1 cross is determined using the described molecular markers. In some embodiments, the resulting F2 progeny is/are screened for any of the low-THCA polymorphisms described herein and provides plants homozygous for the Marker_0 SNP and presenting the low-THC trait.

The plants at any generation can be produced by asexual means like cutting and cloning, or any method that yields a genetically identical offspring.

### Production of Low-THCA Cannabis

In some embodiments, a high-THCA plant may be converted into a low-THCA plant by providing a breeding population where the donor parent plant contains the low-THCA trait and recipient parent plant contains high THCA. In some embodiments the recipient plant comprises one or more other characteristics of interest. For example, high-THC recipient parent plants may be previously characterized and known to exhibit other commercially desirable traits such as, but not limited to, high non-THC cannabinoid concentrations, high biomass, or favorable aroma among other traits. The parent plants are then crossed as described herein.

In some embodiments, the recipient parent plant used in the creation of the breeding population does not contain the low-THCA haplotype. In some embodiments the recipient plant does not contain one or more of the polymorphisms provided in Table 2. In some embodiments the recipient plant does not contain the Marker_0 SNP of THCAS_{G1064A}. In some embodiments the recipient parent plant contains >0.1% THCA in the dry mass of mature inflorescence.

In some embodiments, a low-THC plant obtained through this method will contain one or more traits provided by the recipient parent plant. More particularly, in some embodiments a low-THC plant obtained will contain the majority of the traits provided by the recipient parent plant but will contain the low-THC-trait which has been provided by the donor parent plant.

In some embodiments, the plants identified within the breeding population at either the F1, F2 or later generations, as containing any low-THCA polymorphisms, such as any of the low-THC polymorphisms listed in Table 2 in a heterozygous state, do not contain less than 0.1% THC in the dry weight of the mature inflorescence. In some embodiments, the plants identified within the breeding population at either the F1, F2 or later generations, as containing any low-THCA polymorphisms, such as one or more of the Marker_0 polymorphisms listed in Table 2 in a heterozygous state, do not contain less than 0.1% THC in the dry weight of the mature inflorescence.

In some embodiments, plants containing any low-THCA polymorphisms listed in Table 2, such as the Marker_0 SNP, in a homozygous state contain less than 0.1% THC in the dry weight of the mature inflorescence.

In some embodiments the low-THC phenotype may be introduced into a recipient parent plant by crossing it with a donor parent plant comprising a low-THC phenotype. In some embodiments the donor parent plant comprising a low-THC phenotype comprises one or more of the polymorphisms of Table 2. In some embodiments the donor plant comprises the Marker_0 polymorphism. In some embodiments the recipient parent plant will contain desirable traits e.g. high CBDA content, unique terpene profiles etc. In some embodiments, the donor parent plant is cross fertile with the recipient parent plant.

In some embodiments, MAS or MAB may be used in a method of backcrossing plants carrying the low-THCA trait to a recipient parent plant. In some embodiments the low-THCA plants comprise one or more of the polymorphisms of Table 2. In some embodiments the low-THCA plants comprise the Marker_0 polymorphism. For example, an F1 plant from a breeding population can be crossed again to the recipient parent plant. Offspring, screened using the molecular markers described herein, are identified as containing the low-THCA haplotype and are further enriched for one or more desirable characteristics of the recipient parent plant. In some embodiments, this method is repeated until all of the desired traits from the recipient parent plant are present in one or more plants. A final selfing of the progeny will allow for the low-THCA trait to be selected when homozygous. Cannabis plants developed according to these methods derive the majority of their desired traits from the recipient parent plant, with the low-THCA phenotype from the donor parent plant.

In some embodiments, the resulting plant population is then screened for the low-THC trait using MAS with the described molecular markers to identify progeny plants that contain one or more low-THCA polymorphisms, such as those described in Table 2, for example, the THCAS_{G1064A} SNP Marker_0, indicating a low-THCA phenotype. In another embodiment, the population of Cannabis plants may be screened by measuring cannabinoids directly or by other analytical methods known in the art, e.g. THCA synthase protein activity assays or RT-PCR expression analysis, or by a combination of such methods to identify plants with desired characteristics.

### Production of High-CBGA Cannabis or High CBDA: THCA Ratio Cannabis

Any plant may be converted into a high-CBGA (CBGA dominant) plant according to the methods disclosed herein. In some embodiments a plant may be considered to be dominant for a particular cannabinoid if that cannabinoid makes up greater than 80% of the cannabinoid content of the plant. For example, in some embodiments a CBGA dominant plant may be one in which CBGA makes up greater than 80% of the total cannabinoid content. CBGA is the precursor of THCA, but also of CBDA and CBCA. CBGA will accumulate in plants where there is no, or limited, activity of THCAS, CBDAS, or CBCAS. In some embodiments such a plant is the result of a cross of a donor plant, where the THCAS is inactive (containing the Marker_0 SNP or any of the polymorphisms described herein), with a THC dominant recipient plant, provided it lacks a homozygous Marker_0 SNP, or the polymorphisms described in SEQ ID NOs: 4-216 in said recipient plant. In some embodiments a THC dominant plant may be one in which THC makes up greater than 80% of the total cannabinoid content. Thus, it is possible to create a plant that has an inactive THCAS from the donor parent plant, and a low-activity CBDAS and/or CBCAS from the donor and/or recipient parent plant, leading to the excessive accumulation of CBGA in the progeny, as shown herein. In some embodiments a plant may be converted into a CBDA or CBCA dominant plant that contains the low-THCA phenotype. A plant may be selected for significant CBDAS or CBCAS activity by providing a donor plant containing one or more of the markers of Table 2 and crossing it with a recipient parent plant that contains an active THCAS and an active CBDAS and/or CBCAS. In some embodiments a plant may be selected for significant CBDAS or CBCAS activity by providing a donor plant containing the Marker_0 and crossing it with a recipient parent plant that contains an active THCAS and an active CBDAS and/or CBCAS. A plant may be selected for significant CBDAS or CBCAS activity by providing a donor plant containing the grTHC1.1 region and crossing it with a recipient parent plant that contains an active THCAS and an active CBDAS and/or CBCAS. Thus, it is possible to create a plant that has an inactive THCAS from the donor parent plant, and a high-activity CBDAS and/or CBCAS from the recipient parent plant, leading to the excessive accumulation of CBDA or CBCA in the progeny, as shown herein.

In some embodiments, the recipient parent plant used in the creation of the breeding population does not contain the low-THCA haplotype. In some embodiments the recipient plant does not contain one or more of the low-THCA polymorphisms described in Table 2. In some embodiments the recipient parent plant does not contain the Marker_0 SNP. In some embodiments the recipient plant contains >0.1% THCA and <0.1% CBDA and <0.1% CBCA in the dry mass of mature inflorescence. In another embodiment the recipient plant contains >0.1% THCA and >1% CBDA and/or >1% CBCA in the dry mass of mature inflorescence.

In some embodiments, a high-CBGA plant obtained through the disclosed methods will contain one or more traits provided by the recipient parent plant. More particularly, in some embodiments the high-CBGA plant obtained will contain the majority of the traits provided by the recipient parent plant (e.g. low CBDA and low CBCA levels) except for the low-THC-trait which is provided by the donor parent plant. In another embodiment the high-CBDA and/or high-CBCA plant obtained through this method will contain the low-THCA trait.

In some embodiments, plants are identified within the breeding population at either the F1, F2 or later generations, as containing any of the low-THCA polymorphisms of Table 2 in a heterozygous state, for example the Marker_0 SNP, and do not contain less than 0.1% THC in the dry weight of the mature inflorescence. In some embodiments plants are provided that contain any of the low-THCA polymorphisms and the recited levels of THC.

In some embodiments, plants containing any of the low-THCA polymorphisms described herein, such as the Marker_0 SNP, in a homozygous state contain less than 0.1% THC in the dry weight of the mature inflorescence.

The high-CBGA phenotype may be introduced into a recipient parent plant by crossing it with the donor parent plant (containing any of the polymorphisms described herein). In some embodiments the recipient parent plant will contain desirable traits e.g. low CBDA concentrations, high biomass, unique terpene profiles etc. The donor parent plant is a plant that is cross fertile with the recipient parent plant.

In some embodiments, MAS or MAB may be used in a method of backcrossing plants carrying the low-THCA trait to a recipient parent plant. For example, an F1 plant from the breeding population can be crossed again to the recipient parent plant. Offspring, screened using the molecular markers described herein, are identified as containing the low-THCA haplotype and are further enriched for desirable characteristics of the recipient parent plant. In some embodiments, this method is repeated until all of the desired traits from the recipient parent plant are present in one or more plants. A final selfing of the progeny will allow for the high-CBG trait to be selected when the plant is homozygous for the haplotype described herein. Cannabis plants developed according to these methods derive the majority of their desired traits from the recipient parent plant, and derives the high-CBGA phenotype as a combined result of an inactive THCAS from the donor parent, and a very low activity CBDAS from the recipient parent. In another embodiment, moderate to high CBDAS activity is derived from the recipient plant while the low-THCAS trait is derived from the donor resulting in a plant with an increased ratio of CBDAS to THCAS than observed in the recipient variety.

In some embodiments, the resulting plant population is then screened for the high-CBGA trait using MAS with the described molecular markers to identify progeny plants that contain one or more of the low-THCA polymorphisms, for example, the Marker_0 SNP, indicating a potential high-CBGA phenotype. In another embodiment, the resulting population of Cannabis plants may be screened by measuring cannabinoids directly or by other analytical methods known in the art, e.g. THCA synthase protein activity assays or RT-PCR expression analysis, or by a combination of such methods.

### Methods to Genetically Engineer Plants to Achieve Low-THCA Using Mutagenesis or Gene Editing Techniques

Identifying genomic regions, and individual polymorphisms, that correlate with a trait when measured in an F2, or similar, breeding population indicates the presence of the causative polymorphism in close proximity to, or at the site of, the polymorphism detected by the molecular marker. Polymorphisms in genomic sequences can be introduced by other means so that a trait, such as the low-THC trait, can be introduced into plants that would not otherwise contain associated causative polymorphisms.

One or more of the low-THCA polymorphisms disclosed herein, such as the THCAS_{G1064A} Marker_0 SNP, may be introduced into the genome of a Cannabis plant. In some embodiments the one or more low-THCA polymorphisms is introduced into the genome of a high-THCA plant. The introduction of the low-THCA polymorphism provides the resultant plants or plant parts with a low-THC phenotype. In some embodiments, plants are modified through a process of genetic modification known in the art, for example, but not limited to: CRISPR-Cas9 targeted gene editing, heterologous gene expression using various expression cassettes; TILLING, non-targeted chemical mutagenesis using e.g. EMS.

In some embodiments, plants are provided by manipulating the functional THCAS sequence in any Cannabis variety. In some embodiments the manipulation causes the THCAS to become non-functional or even absent. In some embodiments the THCAS sequence can be altered by targeting and modifying one or more of the nucleotides corresponding to the low-THCA polymorphisms disclosed herein.

In some embodiments, nucleotides of a THCAS gene, homologous to the sequence disclosed in FIG. 3 (SEQ ID NO:1) but lacking the Marker_0 SNP, are modified in any way as to cause one or more amino acid changes that render the THCAS protein defective. In some embodiments the Marker_0 SNP is specifically introduced into the THCAS gene.

In some embodiments low-THC or THC-free plants are provided by partially or entirely silencing a THCAS gene. In some embodiments the THCAS gene is homologous to the sequence disclosed in FIG. 3 (SEQ ID NO:1) but lacking the Marker_0 SNP. In some embodiments the gene is silenced by methods which interfere with transcription of translation of the gene. In some embodiments, silencing is the result of introducing one or more of the polymorphisms from Table 2, such as the Marker_0 SNP.

In some embodiments of the invention, the DNA sequence targeted for modification is the gDNA of a plant, or transcribed cDNA or RNA, or de novo synthesized DNA sequences, or PCR amplicons created used the aforementioned as substrates.

In some embodiments a high-THC variety is genetically modified to contain one or more of the low-THCA polymorphisms, such as the Marker_0 SNP, or THCAS_{C998G} SNP. Plants may be screened with molecular markers as described herein to identify transgenic individuals with a low-THCA polymorphism, such as the Marker_0 SNP, or THCAS_{C998G} SNP.

In some embodiments, Cannabis plants comprising one or more of the polymorphisms of Table 2 are provided. In some embodiments the plants comprise two, three, four, five or more of the polymorphisms of Table 2. In some embodiments the one or more polymorphisms are introduced into the plants. For example, the one or more polymorphisms may be introduced into the plants by genetic engineering. In some embodiments the one or more polymorphisms are introduced into the plants by breeding, such as by MAS or MAB, for example as described herein.

In some embodiments plants comprising the Marker_0 SNP are provided.

In some embodiments Cannabis sativa plants comprising a mutant THCAS enzyme as provided herein, or an isolated nucleic acid as provided herein. In some embodiments Cannabis plants comprising a mutant THCAS or an isolated nucleic acid as provided herein, are provided, with the proviso that the plant is not exclusively obtained by means of an essentially biological process.

In some embodiments plant extracts may be obtained from a Cannabis sativa plant as provided herein. In some embodiments the plant extract has a THCA content of less than 0.1%. In some embodiments the plant extract contains >0.1% THCA and/or <0.1% CBDA and/or <0.1% CBCA. In some embodiments, the plant extract contains >0.1% THCA and >1% CBDA and/or >1% CBCA.

In some embodiments a plant extract obtained from a Cannabis plant as provided herein has the unique Cannabinoid and Terpene profile as shown in Table 1.

In further embodiments, the plant extract provided herein may be used therapeutically, for example in the treatment of cancer, pain, infection, inflammation, Glaucoma and/or cardiovascular diseases. In further embodiments, the plant extract is provided for non-medical use, for example recreational use.

The following examples are offered by way of illustration and not by way of limitation.

### EXAMPLE 1

### Identification of a Genomic Region and Specific Polymorphisms Associated with the Low-THC Phenotype

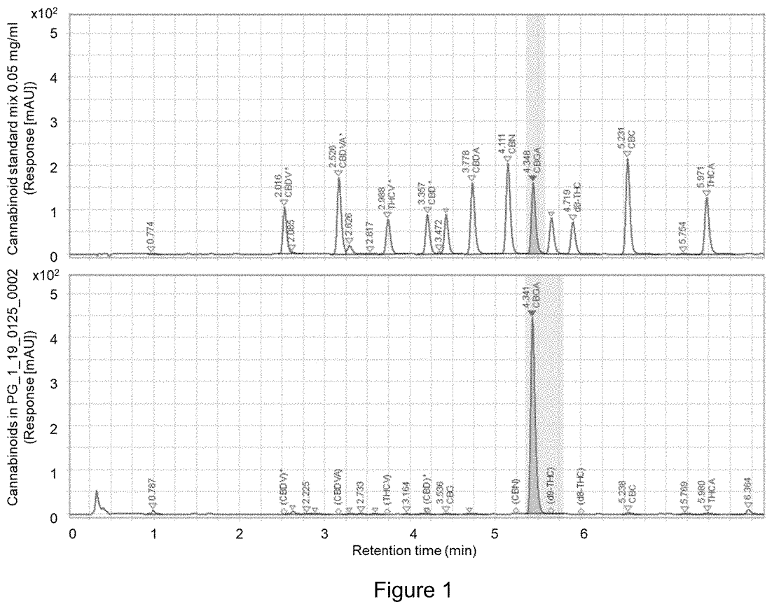

The THCAS gene has been previously associated with the production of THCA in Cannabis sativa. The germplasm collection, including plants that have been subjected to breeding processes, held by Puregene AG was screened for cannabinoid production in the inflorescence by ultra performance liquid chromatography (UPLC). Some varieties were shown to accumulate CBGA up to 10% dry weight, with virtually undetectable THCA concentrations (Table 1). A plant that comprises a genomic region (grTHC1.1) responsible for a low-THC phenotype was identified and is designated PG_1_19_0125_0002. This variety displays a phenotype of <0.1% THCA in the dry weight of the mature inflorescence (FIG. 1). PG_1_19_0125_0002 was thus used herein to identify the genetic mechanism for low THC production.

Analysis of the pangenomic sequences and the genome sequencing of PG_1_19_0125_0002 was used to identify a genomic region associated with the low-THC phenotype. In short, genome sequences were generated for a subset of the plants in the Puregene germplasm collection. The pangenome that was created contained the fully phased, ordered and assembled genomes of a number of sequenced varieties. The genome of the PG_1_19_0125_0002 variety was sequenced as one of the whole genome sequenced varieties using short read Illumina sequencing. The reads were assembled into short contigs and these contigs were used to find the most appropriate reference genome amongst the pangenome collection.

The Assembled Reference Genome 3 (ARG3) was used for this purpose and used as the reference for the assembly of the PG_1_19_0125_0002 genomic reads around a genomic region encompassing the Marker_0 SNP within THCAS_{G1064A} (SEQ ID NO:1). This genomic region is currently called grTHC1.1. This assembly provides a series of polymorphisms (Table 2) contained within PG_1_19_0125_0002 which are able to characterize the grTHC1.1 genomic region. The polymorphisms in Table 2 are only those that are homozygous in PG_1_19_0125_0002 and thus are present in any progeny of PG_1_19_0125_0002, unless recombination occurs between them.

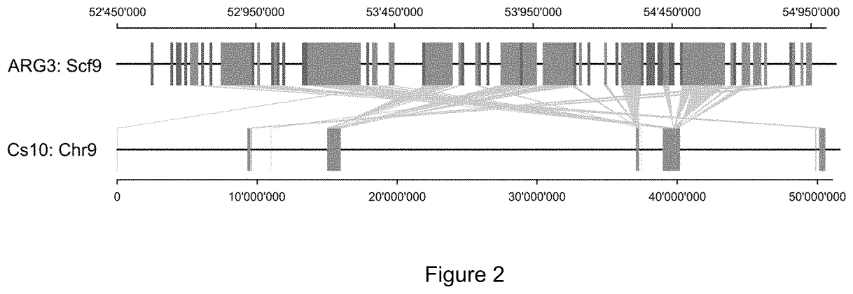

Due to the genetic diversity of the Cannabis species, the pangenomic sequences and that of the PG_1_19_0125_0002 variety were also aligned to the publicly available reference genome of cs10 (FIG. 2 and Table 2). FIG. 2 shows the 2.5 Mb genomic region of ARG3 corresponding to the same region in PG_1_19_0125_0002. This region was mapped to the cs10 genome and a region of 50 Mb of the cs10 chromosome 9 is shown. From FIG. 2 it can be seen that not only are there significant genome rearrangements in PG_1_19_0125_0002 and ARG3 compared to cs10 but also regions that map to entirely different chromosomes (dark grey blocks).

PG_1_19_0125_0002 was identified as being homozygous for a THCAS which was hypothesized to be a non-functional form of the gene.

Using a de novo assembly of whole genome sequences produced for PG_1_19_0125_0002, the THCAS genes contained in the genome were compared to all publicly available sequences (FIG. 3), as well as all sequences of the pangenome.

The inventors found that the THCAS_{G1064A} gene encoded in PG_1_19_0125_0002 contains a number of SNPs as shown in FIG. 3, one of which, “Marker_0”, was completely unique.

Comparison of the nucleotide sequences around the THCAS_{G1064A} revealed that the closest relative of PG_1_19_0125_0002 was ARG3, a plant with a fully assembled genome which forms part of the pangenome. The genomic sequence of PG_1_19_0125_0002 was then assembled to the ARG3 assembly to discover the genomic region denoted as grTHC1.1.

Within grTHC1.1 a number of polymorphisms were detected by comparison to the pangenome as a whole. In Table 2 the polymorphisms which are homozygous in PG_1_19_0125_0002 from this analysis are shown and compared to ARG3, the closest relative.

TABLE 1
Cannabinoids measured in the flowers of PG_1_19_0125_0002
using HPLC coupled to DAD. Percentages represent cannabinoids
content in the dry mass of the flower. Analytes below the
limit of detection are indicated an non detectable (ND).
The UV purity represents the likelihood that a peak is
composed of a single spectroscopic signature, by
comparison with the peak signature of a pure standard.

| | RT | | | Content | UV |
|---|---|---|---|---|---|
| Name | (min) | Area | Amount | (w/w) | Purity |
| CBG | 3.536 | 4.637 | 0.00154 mg | 0.06% | 999.15 |
| CBDA | 3.751 | 6.832 | 0.00148 mg | 0.05% | 999.34 |
| CBGA | 4.341 | 1333.62 | 0.30448 mg | 10.87% | 999.98 |
| CBC | 5.238 | 10.296 | 0.00152 mg | 0.05% | 999.43 |
| THCA | 5.98 | 8.452 | 0.00218 mg | 0.08% | 999.35 |
| CBDV | ND | ND | ND | ND | ND |
| CBDVA | ND | ND | ND | ND | ND |
| CBN | ND | ND | ND | ND | ND |
| d8-THC | ND | ND | ND | ND | ND |
| d9-THC | ND | ND | ND | ND | ND |
| THCV | ND | ND | ND | ND | ND |
| CBD | ND | ND | ND | ND | ND |

RT: Retention time,
CBG: cannabigerol,
CBDA: cannabidiolic acid,
CBGA: cannabigerolic acid,
CBC: cannabichromene,
THCA: tetrahydrocannabinolic acid,
CBDV: cannabidivarin,
CBDVA: cannabidivarinic acid,
CBN: cannabinol,
d8-THC: delta-8-tetrahydrocannabinol,
d9-THC: delta-9-tetrahydrocannabinol,
THCV: tetrahydrocannabivarin,
CBD: cannabidiol

TABLE 2
Genomic region (grTHC1.1) of PG_1_19_0125_0002 assembled to the genome
of ARG3 showing only homozygous polymorphisms in PG_1_19_0125_0002
compared with ARG3. The molecular markers are shown corresponding to
the polymorphism they detect. Polymorphism positions are shown as that
of the ARG3 reference “Ref” and their predicted position on the public
cs10 genome. The variant sequence shows the allele occurring in
PG_1_19_0125_0002 at the same locus (underlined). All of the
polymorphisms occur on scaffold_000009.

| | | | cs10 | | PG | ARG3 | |
|---|---|---|---|---|---|---|---|
| Marker | Position | cs10 chr. | position | Ref | Allele | Allele | Variant |
| MU123 | 52446561 | NA | NA | T | 0/0 | 0/1 | TAAGCACTACAGGGGCACGTACG |
| | | | | | | | SEQ ID NO: 6 |
| MU122 | 52449432 | NA | NA | — | 0/0 | 0/1 | AAAATAAATACTTTTTTTTTTGT |
| | | | | | | | SEQ ID NO: 7 |
| MU121 | 52476023 | NA | NA | G | 1/1 | 0/1 | TTAGGGGTGGACTATATTCCACA |
| | | | | | | | SEQ ID NO: 8 |
| MU120 | 52495493 | NA | NA | C | 0/0 | 0/1 | ATTAAAGAATGTATGCCTTTGCT |
| | | | | | | | SEQ ID NO: 9 |
| MU119 | 52534559 | NA | NA | G | 0/0 | 0/1 | GATACAAATAATATTGGACAGAA |
| | | | | | | | SEQ ID NO: 10 |
| MU118 | 52617621 | NC_044378.1 | 29441682 | T | 0/0 | 0/1 | GTAGTCAAGTTCAGATTCAAAGC |
| | | | | | | | SEQ ID NO: 11 |
| MU117 | 52618467 | NC_044378.1 | 29442528 | A | 0/0 | 0/1 | GATTACTTTACTAAGTGGACTGA |
| | | | | | | | SEQ ID NO: 12 |
| MU116 | 52620188 | NC_044378.1 | 29444248 | A | 0/0 | 0/1 | TGACCTTCTTCTTCAACCTGGCC |
| | | | | | | | SEQ ID NO: 13 |
| MU115 | 52620745 | NC_044378.1 | 29444805 | T | 0/0 | 0/1 | ATGTAATGGGCCAGCATCTCCTT |
| | | | | | | | SEQ ID NO: 14 |
| MU114 | 52622353 | NC_044378.1 | 29447732 | A | 0/0 | 0/1 | ACTACCAGGACGTACATCATCCT |
| | | | | | | | SEQ ID NO: 15 |
| MU113 | 52625421 | NA | NA | A | 0/0 | 0/1 | TCTAAGATCAACTAGTGATATTG |
| | | | | | | | SEQ ID NO: 16 |
| MU112 | 52629922 | NA | NA | G | 0/0 | 0/1 | GGAAGGATAATCTGTATGTTATT |
| | | | | | | | SEQ ID NO: 17 |
| MU111 | 52637405 | NC_044378.1 | 29460797 | — | 0/0 | 0/1 | GTAGTGATTTTGGAGAAGTGTAA |
| | | | | | | | SEQ ID NO: 18 |
| MU110 | 52644340 | NA | NA | A | 0/0 | 0/1 | CACTGTTGACTGAAAATAAGCTA |
| | | | | | | | SEQ ID NO: 19 |
| MU109 | 52690162 | NA | NA | C | 1/1 | 0/1 | TTGATTAACTGTCTTATTTAGTG |
| | | | | | | | SEQ ID NO: 20 |
| MU108 | 52697674 | NC_044378.1 | 44712211 | A | 0/0 | 0/1 | AAATACATAAGGAAGCCAAATAT |
| | | | | | | | SEQ ID NO: 21 |
| MU107 | 52703319 | NA | NA | A | 0/0 | 0/1 | ATTATAACAATGGATAACTAATG |
| | | | | | | | SEQ ID NO: 22 |
| MU106 | 52704011 | NA | NA | T | 0/0 | 0/1 | TCTTTTCTTCTCTTAAAATTTTG |
| | | | | | | | SEQ ID NO: 23 |
| MU105 | 52710980 | NA | NA | A | 0/0 | 0/1 | TCTTGTTCATAGTGGTTTTGACA |
| | | | | | | | SEQ ID NO: 24 |
| MU104 | 52711660 | NC_044378.1 | 44696409 | T | 0/0 | 0/1 | CACAACCGAAGAGGTCAATACCA |
| | | | | | | | SEQ ID NO: 25 |
| MU103 | 52732240 | NA | NA | C | 1/1 | 0/1 | TGTAAAACCCATAATGGAGATCG |
| | | | | | | | SEQ ID NO: 26 |
| MU102 | 52741212 | NA | NA | G | 1/1 | 0/1 | TAACGTGATAAAATTATATGATC |
| | | | | | | | SEQ ID NO: 27 |
| MU101 | 52744565 | NC_044378.1 | 44639629 | A | 0/0 | 0/1 | ACACAGATATCGAGGAAATCCTT |
| | | | | | | | SEQ ID NO: 28 |
| MU100 | 52744698 | NA | NA | T | 0/0 | 0/1 | GTATTATATTTATCCAGGATCTT |
| | | | | | | | SEQ ID NO: 29 |
| MU99 | 52747279 | NC_044378.1 | 44638865 | C | 1/1 | 0/1 | GACAAGAAATTTATTACCAGTAT |
| | | | | | | | SEQ ID NO: 30 |
| MU98 | 52748080 | NC_044378.1 | 44638518 | C | 1/1 | 0/1 | TGCAGTCTTTGTTGCTTCAGTCT |
| | | | | | | | SEQ ID NO: 31 |
| MU97 | 52753492 | NA | NA | G | 0/0 | 0/1 | CATTAGTGTAAATCCAATAACAT |
| | | | | | | | SEQ ID NO: 32 |
| MU96 | 52768005 | NC_044378.1 | 44630317 | G | 1/1 | 0/1 | ATCTAGATCCTAGTAAAATAATT |
| | | | | | | | SEQ ID NO: 33 |
| MU95 | 52781075 | NA | NA | C | 1/1 | 0/1 | TATAAATCAAATAGACAAACAAT |
| | | | | | | | SEQ ID NO: 34 |
| MU94 | 52783456 | NA | NA | G | 1/1 | 0/1 | TGCTTTAGGCAATATATTGGGAT |
| | | | | | | | SEQ ID NO: 35 |
| MU93 | 52785212 | NA | NA | C | 1/1 | 0/1 | TAAGTCTTATA-AGTCGCCACCA |
| | | | | | | | SEQ ID NO: 36 |
| MU92 | 52787310 | NA | NA | T | 1/1 | 0/1 | CTGTAAAAAAGGAAATTGCTTAG |
| | | | | | | | SEQ ID NO: 37 |
| MU91 | 52789722 | NA | NA | C | 1/1 | 0/1 | TGATGCATTGGAAAAGTTGGCAT |
| | | | | | | | SEQ ID NO: 38 |
| MU90 | 52798433 | NA | NA | C | 1/1 | 0/1 | ATCTGGCCGACAACTCCCTCTCT |
| | | | | | | | SEQ ID NO: 39 |
| MU89 | 52799431 | NC_044378.1 | 44599558 | C | 1/1 | 0/1 | TGAAATAAAACGTGGCAGCTCTG |
| | | | | | | | SEQ ID NO: 40 |
| MU88 | 52800244 | NA | NA | G | 1/1 | 0/1 | GGGAATATTGAAAAAATAATTTT |
| | | | | | | | SEQ ID NO: 41 |
| MU87 | 52802281 | NC_044378.1 | 44596684 | C | 1/1 | 0/1 | CATGATATTTTTAAGTCAAAAAT |
| | | | | | | | SEQ ID NO: 42 |
| MU86 | 52802955 | NA | NA | C | 1/1 | 0/1 | AAACCCTAGTAGCATGTCCAAAA |
| | | | | | | | SEQ ID NO: 43 |
| MU85 | 52804828 | NA | NA | C | 1/1 | 0/1 | CCATTGGCTCCTAGATGCTTGAT |
| | | | | | | | SEQ ID NO: 44 |
| MU84 | 52805347 | NA | NA | C | 1/1 | 0/1 | ACGCCAACTTTGGTATCCATGAT |
| | | | | | | | SEQ ID NO: 45 |
| MU83 | 52807733 | NA | NA | G | 1/1 | 0/1 | ATAAAAACGATAATTTATATGTT |
| | | | | | | | SEQ ID NO: 46 |
| MU82 | 52828304 | NA | NA | A | 0/0 | 0/1 | TATCACCTGTTGATCCTAGATCA |
| | | | | | | | SEQ ID NO: 47 |
| MU81 | 52833724 | NA | NA | A | 1/1 | 0/1 | AGGTACTATTAGTTTTAAATTGT |
| | | | | | | | SEQ ID NO: 48 |
| MU80 | 52833892 | NC_044378.1 | 44578488 | A | 0/0 | 0/1 | AAATTAATAAAGCAAAATAAAGA |
| | | | | | | | SEQ ID NO: 49 |
| MU79 | 52838069 | NA | NA | A | 0/0 | 0/1 | ATAGTAGAGGGGATTAGAATCCA |
| | | | | | | | SEQ ID NO: 50 |
| MU78 | 52838371 | NA | NA | G | 0/0 | 0/1 | AATGGATCCATAGATAAATATTT |
| | | | | | | | SEQ ID NO: 51 |
| MU77 | 52855321 | NA | NA | T | 0/0 | 0/1 | CACAAAACATGCAAAATTATGAA |
| | | | | | | | SEQ ID NO: 52 |
| MU76 | 52859285 | NA | NA | A | 0/0 | 0/1 | TCAATATCGGTCAAGTGCAATGT |
| | | | | | | | SEQ ID NO: 53 |
| MU75 | 52859886 | NA | NA | A | 0/0 | 0/1 | CTTTTAGGTCAGGGTGATACGTA |
| | | | | | | | SEQ ID NO: 54 |
| MU74 | 52866094 | NA | NA | A | 1/1 | 0/1 | TTCTTCGTTGAGTCCACGCATAA |
| | | | | | | | SEQ ID NO: 55 |
| MU73 | 52889600 | NA | NA | T | 0/0 | 0/1 | CACGTTTTGATGAGGTTCCATCT |
| | | | | | | | SEQ ID NO: 56 |
| MU72 | 52890272 | NA | NA | A | 0/0 | 0/1 | GTCCTGTTTAGGCGAGAGTCAAG |
| | | | | | | | SEQ ID NO: 57 |
| MU71 | 52895125 | NA | NA | T | 1/1 | 0/1 | AATAAATCATGAGAACCATGCAA |
| | | | | | | | SEQ ID NO: 58 |
| MU70 | 52900670 | NC_044378.1 | 29473999 | T | 0/0 | 0/1 | TATTTATTTTTCGAAAATTTAAA |
| | | | | | | | SEQ ID NO: 59 |
| MU69 | 52904471 | NC_044378.1 | 29476845 | T | 0/0 | 0/1 | TATATTAATAAGTAAATCTCATT |
| | | | | | | | SEQ ID NO: 60 |
| MU68 | 52909498 | NC_044378.1 | 29481868 | G | 1/1 | 0/1 | TAAGAGTTGTTCACTGAGAGCTG |
| | | | | | | | SEQ ID NO: 61 |
| | | | | | | | AAGGTTTACAAGCACAACCCTAT |
| MU67 | 52911866 | NA | NA | C | 1/1 | 0/1 | SEQ ID NO: 62 |
| | | | | | | | GAATAAGGAATCCTCATCTCCTT |
| MU66 | 52912625 | NC_044378.1 | 29485095 | G | 1/1 | 0/1 | SEQ ID NO: 63 |
| | | | | | | | ATACTCCTTCAGTTGTCTGGATG |
| MU65 | 52915979 | NA | NA | A | 1/1 | 0/1 | SEQ ID NO: 64 |
| | | | | | | | CTCATACCAATATGTTTACTCAT |
| MU64 | 52919386 | NA | NA | G | 0/0 | 0/1 | SEQ ID NO: 65 |
| | | | | | | | ATAATCTGAACTCGAAGGAGAAG |
| MU63 | 52923290 | NC_044378.1 | 29499709 | C | 1/1 | 0/1 | SEQ ID NO: 66 |
| | | | | | | | TGTAAGTCTTATACAGTCGCCAC |
| MU62 | 52938359 | NA | NA | C | 0/0 | 0/1 | SEQ ID NO: 67 |
| | | | | | | | TACTCCAAGTGTAAGTATACTTC |
| MU61 | 52943420 | NA | NA | A | 0/0 | 0/1 | SEQ ID NO: 68 |
| | | | | | | | TAAATTGATAACTAATCAGATTA |
| MU60 | 52943650 | NC_044378.1 | 29520276 | A | 0/0 | 0/1 | SEQ ID NO: 69 |
| | | | | | | | TTTCCTGTTCCCTGGTTTATGGC |
| MU59 | 52945607 | NC_044378.1 | 29522195 | A | 0/0 | 0/1 | SEQ ID NO: 70 |
| | | | | | | | CCTCCCCTCTGGCCAAGAAGTCT |
| MU58 | 52949561 | NC_044377.1 | 21727324 | A | 0/0 | 0/1 | SEQ ID NO: 71 |
| | | | | | | | ACGATGATGTATCGTGCTGCCTT |
| MU57 | 52952494 | NA | NA | C | 0/0 | 0/1 | SEQ ID NO: 72 |
| | | | | | | | ATAATCTTACCATACAGGGATTT |
| MU56 | 53011869 | NA | NA | C | 1/1 | 0/0 | SEQ ID NO: 73 |
| | | | | | | | TCGTCTTTCAGTGCTACTGCTCG |
| MUSS | 53049693 | NA | NA | G | 1/1 | 0/1 | SEQ ID NO: 74 |
| | | | | | | | CCATTGCTGTGTGGCCTCTAACA |
| MU54 | 53051528 | NA | NA | C | 1/1 | 0/1 | SEQ ID NO: 75 |
| | | | | | | | GTTTTGGAGTTGAAATTCTGCAT |
| MU53 | 53053532 | NA | NA | A | 1/1 | 0/1 | SEQ ID NO: 76 |
| | | | | | | | CATTCAAATACACATTAATATAA |
| MU52 | 53054332 | NA | NA | T | 1/1 | 0/1 | SEQ ID NO: 77 |
| | | | | | | | GGCTGAATAATCTAAAGTGGCCA |
| MU51 | 53065049 | NA | NA | T | 0/0 | 0/1 | SEQ ID NO: 78 |
| | | | | | | | GCTCAACTCAACGGATCACTAAT |
| MU50 | 53079563 | NA | NA | T | 0/0 | 0/1 | SEQ ID NO: 79 |
| | | | | | | | TAGACTGGATGCTGCACGCCATC |
| MU49 | 53084276 | NA | NA | T | 0/0 | 0/1 | SEQ ID NO: 80 |
| | | | | | | | CTGCAAGCCAAATTTCTTGGAGC |
| MU48 | 53091632 | NA | NA | C | 1/1 | 0/1 | SEQ ID NO: 81 |
| | | | | | | | GTTGTTTTATGTCGCGAACAACA |
| MU47 | 53114128 | NA | NA | C | 0/0 | 0/1 | SEQ ID NO: 82 |
| | | | | | | | AGAGACAGAAAAAGAAGACCCTT |
| MU46 | 53118124 | NA | NA | G | 0/0 | 0/1 | SEQ ID NO: 83 |
| | | | | | | | AAATTGGGGCTATGCTGATATTT |
| MU45 | 53120394 | NA | NA | G | 1/1 | 0/1 | SEQ ID NO: 84 |
| | | | | | | | ATTCTAATTATGTTTTTGAATTA |
| MU44 | 53120549 | NA | NA | C | 1/1 | 0/1 | SEQ ID NO: 85 |
| | | | | | | | TAGCTAATCTGCGGCCGACCCAT |
| MU43 | 53135294 | NC_044371.1 | 24686310 | T | 1/1 | 0/1 | SEQ ID NO: 86 |
| | | | | | | | AATGGTGAGCCCTTAATGCCTCC |
| MU42 | 53138951 | NA | NA | T | 0/0 | 0/1 | SEQ ID NO: 87 |
| | | | | | | | GTTTCACTAACAGGAGTTGTCCC |
| MU41 | 53139232 | NC_044378.1 | 29633112 | C | 0/0 | 0/1 | SEQ ID NO: 88 |
| | | | | | | | TAAAGCAAATGAGTGTCTCCCAT |
| MU40 | 53148031 | NA | NA | G | 1/1 | 0/1 | SEQ ID NO: 89 |
| | | | | | | | TGCTCAAATCCCAGAGAATAACA |
| MU39 | 53176808 | NA | NA | T | 0/0 | 0/1 | SEQ ID NO: 90 |
| | | | | | | | ATATATTGTTTATTGAGAGGATA |
| MU38 | 53199152 | NC_044378.1 | 29746266 | G | 1/1 | 0/1 | SEQ ID NO: 91 |
| | | | | | | | AGGCTCAATCGGTCCAAAGAAAA |
| MU37 | 53310731 | NA | NA | A | 0/0 | 0/1 | SEQ ID NO: 92 |
| | | | | | | | TGATACATTTCAGTGGTTCCCGA |
| MU36 | 53379642 | NC_044372.1 | 60782540 | G | 1/1 | 0/1 | SEQ ID NO: 93 |
| | | | | | | | TACACTTTTGACGCCTTTGATGC |
| MU35 | 53390348 | NC_044378.1 | 29788957 | T | 1/1 | 0/1 | SEQ ID NO: 4 |
| | | | | | | | ATCGTTTTGCTTGAAATTTTGTC |
| MU34 | 53392478 | NA | NA | C | 1/1 | 0/1 | SEQ ID NO: 94 |
| | | | | | | | CAAGGGCAAACAGGTAATTACAA |
| MU33 | 53393529 | NC_044378.1 | 2271384 | G | 1/1 | 0/1 | SEQ ID NO: 95 |
| | | | | | | | AAAGGACCTAAAAGTAAAGCTTA |
| MU32 | 53395409 | NC_044378.1 | 2273263 | G | 1/1 | 0/0 | SEQ ID NO: 96 |
| | | | | | | | CTTTGGGAACTGCCCCAATAACT |
| MU31 | 53401668 | NA | NA | A | 0/0 | 0/1 | SEQ ID NO: 97 |
| | | | | | | | TCGACCCCACTAAGTTGCCCTGA |
| MU30 | 53419823 | NA | NA | G | 1/1 | 0/1 | SEQ ID NO: 98 |
| | | | | | | | TGTTCACGACACAGTCACTAATC |
| MU29 | 53459093 | NA | NA | A | 0/0 | 0/1 | SEQ ID NO: 99 |
| | | | | | | | AATGTGATTGTGATCATATGAAT |
| MU28 | 53462422 | NA | NA | T | 0/0 | 0/1 | SEQ ID NO: 100 |
| | | | | | | | TAGTGAAAGAGTAAGTGCCCACA |
| MU27 | 53469920 | NA | NA | C | 0/0 | 0/1 | SEQ ID NO: 101 |
| | | | | | | | CATTTAACAAACTCGCTATTATA |
| MU26 | 53476855 | NC_044379.1 | 21160830 | A | 0/0 | 0/1 | SEQ ID NO: 102 |
| | | | | | | | AAAATCTAATTTTTTGTATACTA |
| MU25 | 53476978 | NC_044379.1 | 21160953 | C | 0/0 | 0/1 | SEQ ID NO: 103 |
| | | | | | | | TTACCAGGTGCACTTTTACCTTT |
| MU24 | 53477375 | NC_044379.1 | 21161351 | G | 0/0 | 0/1 | SEQ ID NO: 104 |
| | | | | | | | TTTTTCATAATCGCAAATTGGCG |
| MU23 | 53477551 | NA | NA | T | 0/0 | 0/1 | SEQ ID NO: 105 |
| | | | | | | | ATTATGCTTATCTAATCGCAAAT |
| MU22 | 53477686 | NA | NA | G | 0/0 | 0/1 | SEQ ID NO: 106 |
| | | | | | | | GATTTTTACCCATTTGCCCTACA |
| MU21 | 53500985 | NA | NA | T | 0/0 | 0/1 | SEQ ID NO: 107 |
| | | | | | | | ATCTGATATAAATTTTATCTTTA |
| MU20 | 53526625 | NA | NA | C | 1/1 | 0/0 | SEQ ID NO: 108 |
| | | | | | | | TCAAATCACTCTAAACTTACTTA |
| MU19 | 53574748 | NA | NA | C | 0/0 | 0/1 | SEQ ID NO: 109 |
| | | | | | | | AATATTTGCTTCAGGTTTTATCG |
| MU18 | 53615693 | NA | NA | A | 0/0 | 0/1 | SEQ ID NO: 110 |
| | | | | | | | TGGAAGAGAACGGAACATTGGAA |
| MU17 | 53622904 | NA | NA | A | 0/0 | 0/1 | SEQ ID NO: 111 |
| | | | | | | | ATACTTAAAATATTGATTCATCC |
| MU16 | 53633091 | NC_044377.1 | 56384833 | G | 1/1 | 0/0 | SEQ ID NO: 112 |
| | | | | | | | TCTAACCCTTGTTTCCTTTCTGT |
| MU15 | 53677933 | NA | NA | A | 1/1 | 0/1 | SEQ ID NO: 113 |
| | | | | | | | CGACATTTTTCTGTGGACTGTCT |
| MU14 | 53683961 | NA | NA | A | 1/1 | 0/1 | SEQ ID NO: 114 |
| | | | | | | | TTATAATGTTGGGTTTTATGCCC |
| MU13 | 53685660 | NA | NA | A | 0/0 | 0/1 | SEQ ID NO: 115 |
| | | | | | | | GCTCTGCTACAGAGGAATCAA |
| MU12 | 53687374 | NA | NA | A | 0/0 | 0/1 | SEQ ID NO: 116 |
| | | | | | | | CACAGACGGCGCCAAATGTTGTC |
| MU11 | 53701681 | NA | NA | T | 0/0 | 0/1 | SEQ ID NO: 117 |
| | | | | | | | TGGCTTTGTATCGAAATTCAGTT |
| MU10 | 53717044 | NA | NA | T | 0/0 | 0/1 | SEQ ID NO: 118 |
| | | | | | | | TATAAGAGGTCGGTTTTGTGTGT |
| MU9 | 53727359 | NA | NA | T | 0/0 | 0/1 | SEQ ID NO: 119 |
| | | | | | | | CTGGTTCCCCCAGAACCTCTTGT |
| MU8 | 53740645 | NA | NA | G | 1/1 | 0/1 | SEQ ID NO: 120 |
| | | | | | | | CCCAGTAACTATATCACCATCAT |
| MU7 | 53748083 | NA | NA | A | 1/1 | 0/1 | SEQ ID NO: 121 |
| | | | | | | | TGGTCAGATCAAAACAACTTTGT |
| MU6 | 53751743 | NA | NA | G | 1/1 | 0/1 | SEQ ID NO: 122 |
| | | | | | | | CAACCAAAACGTTTCCAAACTCA |
| MU5 | 53764842 | NA | NA | A | 0/0 | 0/1 | SEQ ID NO: 123 |
| | | | | | | | CAAACTTTTGTGGAAAAGATACT |
| MU4 | 53782279 | NA | NA | T | 0/0 | 0/1 | SEQ ID NO: 124 |
| | | | | | | | AGATAACCGCTAACTTTAGGGTA |
| MU3 | 53809724 | NA | NA | C | 1/1 | 0/1 | SEQ ID NO: 125 |
| | | | | | | | AGTGTCAAGTCTTCTATGTCACT |
| MU2 | 53816852 | NA | NA | C | 1/1 | 0/1 | SEQ ID NO: 126 |
| | | | | | | | GCCTCGGGTATTGTGCCGACCGA |
| MU1 | 53838510 | NA | NA | C | 0/0 | 0/1 | SEQ ID NO: 127 |
| | | | | | | | TTACAACACCATTGTAGAAGATG |
| M_O | 53870757 | NA | NA | C | 1/1 | 0/0 | SEQ ID NO: 3 |
| | | | | | | | CCATCCCCGCACGACACCAACTG |
| MD1 | 53888015 | NA | NA | T | 1/1 | 0/1 | SEQ ID NO: 128 |
| | | | | | | | AGGCGGACTACTAGGAGTCAGGG |
| MD2 | 53899685 | NA | NA | C | 1/1 | 0/1 | SEQ ID NO: 129 |
| | | | | | | | AGTCGTGGCTTTCCATTGCAGCC |
| MD3 | 53930032 | NA | NA | C | 1/1 | 0/1 | SEQ ID NO: 130 |
| | | | | | | | TCTTTAACCATAACCACTTTTTT |
| MD4 | 53930367 | NC_044376.1 | 47538476 | T | 1/1 | 0/0 | SEQ ID NO: 131 |
| | | | | | | | CATTGCAAAATCATAAGACATCA |
| MDS | 53931044 | NA | NA | A | 1/1 | 0/1 | SEQ ID NO: 132 |
| | | | | | | | ATATTTTTATACATTTAACTTTT |
| MD6 | 54000948 | NA | NA | A | 0/0 | 0/1 | SEQ ID NO: 133 |
| | | | | | | | GTACTTGAGTCCAGAGTTAGTGC |
| MD7 | 54046767 | NA | NA | T | 0/0 | 0/1 | SEQ ID NO: 134 |
| | | | | | | | ACATGTTTTCCTGCGGATTTCAC |
| MD8 | 54046902 | NA | NA | C | 0/0 | 0/1 | SEQ ID NO: 135 |
| | | | | | | | TCAACATTCTCTTTTTGGTTTGT |
| MD9 | 54062427 | NC_044378.1 | 30983014 | C | 1/1 | 0/1 | SEQ ID NO: 136 |
| | | | | | | | TGTGATTGTGTTTATCGACGACA |
| MD10 | 54077334 | NA | NA | A | 0/0 | 0/1 | SEQ ID NO: 137 |
| | | | | | | | AATTTCAATGCCCCTAACCGAGC |
| MD11 | 54077637 | NA | NA | G | 0/0 | 0/1 | SEQ ID NO: 138 |
| | | | | | | | GTAGTTTGTCTCATGTTGCTCGC |
| MD12 | 54083234 | NA | NA | A | 0/0 | 0/1 | SEQ ID NO: 139 |
| | | | | | | | CCGGTCATACTCCGGCTGCTAGT |
| MD13 | 54091771 | NA | NA | T | 0/0 | 0/1 | SEQ ID NO: 140 |
| | | | | | | | AATGGACCTAAATAAAATCTATC |
| MD14 | 54106845 | NA | NA | G | 0/0 | 0/1 | SEQ ID NO: 141 |
| | | | | | | | GCTCCATTATATAAAGCTATAAA |
| MD15 | 54150793 | NA | NA | A | 0/0 | 0/1 | SEQ ID NO: 142 |
| | | | | | | | TTACACACAAGAAAGTATTTGGG |
| MD16 | 54152360 | NC_044378.1 | 30981528 | C | 1/1 | 0/1 | SEQ ID NO: 143 |
| | | | | | | | AAAAAAATTGAAGTATATAAGAA |
| MD17 | 54179134 | NA | NA | T | 0/0 | 0/1 | SEQ ID NO: 144 |
| | | | | | | | CCAATACTCATCGTATAACGCCG |
| MD18 | 54189012 | NA | NA | T | 0/0 | 0/1 | SEQ ID NO: 145 |
| | | | | | | | CCATCCCGCCACATCCTGCTTCA |
| MD19 | 54192976 | NA | NA | G | 0/0 | 0/1 | SEQ ID NO: 146 |
| | | | | | | | CTACGCTTTTTGTGTCGTAGAAA |
| MD20 | 54193925 | NA | NA | A | 1/1 | 0/1 | SEQ ID NO: 147 |
| | | | | | | | AACCCCCATAACCTCAGCAATCT |
| MD21 | 54224942 | NA | NA | T | 0/0 | 0/1 | SEQ ID NO: 148 |
| | | | | | | | CTAGGTGCAGCTCAGCCTCTGGC |
| MD22 | 54273162 | NA | NA | C | 1/1 | 0/1 | SEQ ID NO: 149 |
| | | | | | | | TTATTCTCTCCTGAAGATGCTTC |
| MD23 | 54289646 | NA | NA | G | 0/0 | 0/1 | SEQ ID NO: 150 |
| | | | | | | | AACCTTTGCATTAAGAAGGTTTT |
| MD24 | 54289904 | NC_044373.1 | 14667695 | C | 0/0 | 0/1 | SEQ ID NO: 151 |
| | | | | | | | TTCAAAATTTCAAAGTTAGAAAA |
| MD25 | 54312899 | NA | NA | T | 0/0 | 0/1 | SEQ ID NO: 152 |
| | | | | | | | GAGTTTTTGAACTCAATCTTGAA |
| MD26 | 54313384 | NA | NA | A | 0/0 | 0/1 | SEQ ID NO: 153 |
| | | | | | | | TTGTAATTAGTCGCTAGGCGACA |
| MD27 | 54314609 | NA | NA | A | 0/0 | 0/1 | SEQ ID NO: 154 |
| | | | | | | | GCTTTTTAATCGACATGATATCG |
| MD28 | 54326544 | NA | NA | A | 0/0 | 0/1 | SEQ ID NO: 155 |
| | | | | | | | GAATGGCCGGGAATTTAGAATGT |
| MD29 | 54327200 | NA | NA | G | 0/0 | 0/1 | SEQ ID NO: 156 |
| | | | | | | | AAATAAATTAAATTTAATTTTTA |
| MD30 | 54358731 | NA | NA | T | 1/1 | 0/1 | SEQ ID NO: 157 |
| | | | | | | | ATGTAATTTCCATTCCTTAATTG |
| MD31 | 54367396 | NA | NA | T | 0/0 | 0/1 | SEQ ID NO: 158 |
| | | | | | | | GTCTCGAAGAGGACATTGTTGCT |
| MD32 | 54372474 | NA | NA | A | 0/0 | 0/1 | SEQ ID NO: 159 |
| | | | | | | | CTCAATTTCGATTTAATTTGAAT |
| MD33 | 54397927 | NC_044379.1 | 34552052 | A | 0/0 | 0/1 | SEQ ID NO: 160 |
| | | | | | | | TAGCATGCTCACCCTAGTAATCA |
| MD34 | 54405637 | NA | NA | A | 1/1 | 0/0 | SEQ ID NO: 161 |
| | | | | | | | CATTTTTTCATGTTTTGTGCCCG |
| MD35 | 54424908 | NA | NA | A | 0/0 | 0/1 | SEQ ID NO: 162 |
| | | | | | | | CTCCTAATATTCCCTGGCGTCCA |
| MD36 | 54449109 | NA | NA | T | 0/0 | 0/1 | SEQ ID NO: 163 |
| | | | | | | | AATTTCTCTCTCCAGAAAAATTG |
| MD37 | 54453499 | NC_044378.1 | 16779271 | T | 1/1 | 0/1 | SEQ ID NO: 164 |
| | | | | | | | TATCGTAATATATATTCAATTAA |
| MD38 | 54505515 | NA | NA | C | 1/1 | 0/1 | SEQ ID NO: 165 |
| | | | | | | | ACTTTACGCCATATAAATAACTT |
| MD39 | 54538522 | NA | NA | G | 0/0 | 0/1 | SEQ ID NO: 166 |
| | | | | | | | TATAGAGGATCCTTGATCAAATT |
| MD40 | 54562340 | NA | NA | A | 1/1 | 0/1 | SEQ ID NO: 167 |
| | | | | | | | AAATGGCAATAGTATTTAATAAT |
| MD41 | 54562738 | NA | NA | T | 1/1 | 0/1 | SEQ ID NO: 168 |
| | | | | | | | TACTAATTCCAGCCAACGCCTCT |
| MD42 | 54611944 | NA | NA | A | 1/1 | 0/1 | SEQ ID NO: 169 |
| | | | | | | | ATAACTCCTTATCTGACATGGCC |
| MD43 | 54613288 | NA | NA | G | 1/1 | 0/1 | SEQ ID NO: 170 |
| | | | | | | | ATGCTTCCTATAAGGCTTTTCTT |
| MD44 | 54615082 | NA | NA | C | 1/1 | 0/1 | SEQ ID NO: 171 |
| | | | | | | | ACAATGTATCAAAATTAACAAAT |
| MD45 | 54619576 | NA | NA | T | 1/1 | 0/1 | SEQ ID NO: 172 |
| | | | | | | | TTTAGTTTGTTGAATGTTATTTT |
| MD46 | 54626689 | NA | NA | A | 0/0 | 0/1 | SEQ ID NO: 173 |
| | | | | | | | CAAATACATGCCAGGTAAAATTA |
| MD47 | 54626917 | NA | NA | A | 1/1 | 0/1 | SEQ ID NO: 174 |
| | | | | | | | CAATATTCTCCCAAAATTACCCC |
| MD48 | 54649534 | NC_044379.1 | 54971646 | G | 0/0 | 0/1 | SEQ ID NO: 175 |
| | | | | | | | TCACTAAGCATGGACGAAATTGT |
| MD49 | 54681193 | NA | NA | T | 0/0 | 0/1 | SEQ ID NO: 176 |
| | | | | | | | ACTTAAATATATATTTCTAATTA |
| MD50 | 54709190 | NA | NA | A | 0/0 | 0/1 | SEQ ID NO: 177 |
| | | | | | | | AAACAACATTCATCATTTTAAAT |
| MD51 | 54711188 | NA | NA | G | 0/0 | 0/1 | SEQ ID NO: 178 |
| | | | | | | | GTTATATGTCTAAGGTTATATGT |
| MD52 | 54732400 | NA | NA | G | 1/1 | 0/1 | SEQ ID NO: 179 |
| | | | | | | | ATTTTCACTAACCAGGGGTGAAA |
| MD53 | 54733581 | NA | NA | G | 1/1 | 0/1 | SEQ ID NO: 180 |
| | | | | | | | GAATCTCTACTATTGCAGGACAT |
| MD54 | 54740568 | NA | NA | T | 0/0 | 0/1 | SEQ ID NO: 181 |
| | | | | | | | CTCAACATGTACAGAAACAAGTT |
| MD55 | 54747484 | NA | NA | T | 0/0 | 0/1 | SEQ ID NO: 182 |
| | | | | | | | TCCTTCATTTCAGTTACTTTCCA |
| MD56 | 54756843 | NA | NA | G | 1/1 | 0/1 | SEQ ID NO: 183 |
| | | | | | | | GGTACCTGCAGACTGAACCTGTT |
| MD57 | 54774103 | NA | NA | C | 1/1 | 0/1 | SEQ ID NO: 184 |
| | | | | | | | CTTTTAACGACAGGATAATTGTA |
| MD58 | 54776901 | NA | NA | G | 1/1 | 0/1 | SEQ ID NO: 185 |
| | | | | | | | GTAAGAAAAGAGAGAAAAGATAT |
| MD59 | 54790306 | NA | NA | T | 0/0 | 0/1 | SEQ ID NO: 186 |
| | | | | | | | TGAACCATGCAACATTAAATGTT |
| MD60 | 54812445 | NA | NA | G | 0/0 | 0/1 | SEQ ID NO: 187 |
| | | | | | | | GAAAGGGAATTAGTATCAAAAGA |
| MD61 | 54819693 | NC_044378.1 | 30898659 | G | 0/0 | 0/1 | SEQ ID NO: 188 |
| | | | | | | | CACAAACCCGGACTGGGACCAGG |
| MD62 | 54825730 | NC_044378.1 | 30880160 | G | 0/0 | 0/1 | SEQ ID NO: 189 |
| | | | | | | | AGGGTCCGGGTCTGGTCGTAATC |
| MD63 | 54826923 | NC_044378.1 | 30878978 | A | 0/0 | 0/1 | SEQ ID NO: 190 |
| | | | | | | | CATCATCCCCACCACGAACACAA |
| MD64 | 54827785 | NC_044378.1 | 30878117 | T | 0/0 | 0/1 | SEQ ID NO: 191 |
| | | | | | | | AATAGTTGGCATGGAAGAAAGAA |
| MD65 | 54830077 | NC_044378.1 | 30875495 | C | 0/0 | 0/1 | SEQ ID NO: 192 |
| | | | | | | | TTGAAAAATTAACGTTTTTGAGA |
| MD66 | 54832332 | NA | NA | G | 1/1 | 0/1 | SEQ ID NO: 193 |
| | | | | | | | CATTAATTCAGGTGCATCAATGT |
| MD67 | 54832794 | NA | NA | T | 1/1 | 0/1 | SEQ ID NO: 194 |
| | | | | | | | ACTTAACTAACCCCAGTGGAGAG |
| MD68 | 54840146 | NC_044378.1 | 30865475 | T | 0/0 | 0/1 | SEQ ID NO: 195 |
| | | | | | | | TGACTTGAATATATTTTTCGGTA |
| MD69 | 54847470 | NA | NA | G | 1/1 | 0/1 | SEQ ID NO: 196 |
| | | | | | | | ATTAAGGAAGGCAGTCTTGACAT |
| MD70 | 54849140 | NA | NA | T | 0/0 | 0/1 | SEQ ID NO: 197 |
| | | | | | | | AATATAAAACTTTTAGGTTATAA |
| MD71 | 54852980 | NC_044378.1 | 30851503 | G | 1/1 | 0/1 | SEQ ID NO: 198 |
| | | | | | | | TGATTAATCTTTGTTTATTTTCT |
| MD72 | 54854034 | NA | NA | C | 1/1 | 0/1 | SEQ ID NO: 199 |
| | | | | | | | TAATAGATGCTACAGCTGGAGGA |
| MD73 | 54855104 | NA | NA | G | 1/1 | 0/1 | SEQ ID NO: 200 |
| | | | | | | | AACAATCCCAAATTGTTTGTGAG |
| MD74 | 54855311 | NA | NA | G | 1/1 | 0/1 | SEQ ID NO: 201 |
| | | | | | | | TGAAACAGTGGCATTAACTGAGG |
| MD75 | 54856071 | NA | NA | T | 0/0 | 0/1 | SEQ ID NO: 202 |
| | | | | | | | ACCTAAGTGAAATGAGATCTCCA |
| MD76 | 54863786 | NA | NA | G | 0/0 | 0/1 | SEQ ID NO: 203 |
| | | | | | | | ATGAAAAATTATCAAATTAAAGT |
| MD77 | 54866322 | NA | NA | — | 1/1 | 0/1 | SEQ ID NO: 204 |
| | | | | | | | AAAAGCTAGTGGAAAAATGTGCC |
| MD78 | 54873748 | NA | NA | A | 0/0 | 0/1 | SEQ ID NO: 205 |
| | | | | | | | AAGTGCTACTTTTGTGGAAGGGC |
| MD79 | 54880255 | NC_044378.1 | 30824419 | C | 1/1 | 0/1 | SEQ ID NO: 206 |
| | | | | | | | GGGAGAAGTCAGACAGACAGGTC |
| MD80 | 54881920 | NC_044378.1 | 30822750 | A | 0/0 | 0/1 | SEQ ID NO: 207 |
| | | | | | | | TTGGAGTAAGTGTTATGTTCTTT |
| MD81 | 54882393 | NA | NA | A | 0/0 | 0/1 | SEQ ID NO: 208 |
| | | | | | | | TTTATTTTTCGAAAAAATAAATT |
| MD82 | 54885152 | NA | NA | — | 1/1 | 0/1 | SEQ ID NO: 209 |
| | | | | | | | TATTTTAGATACTTTTTAGGTTA |
| MD83 | 54886181 | NA | NA | T | 1/1 | 0/0 | SEQ ID NO: 210 |
| | | | | | | | ATTGGTCGTTCTTTTCACATTGA |
| MD84 | 54888497 | NA | NA | C | 1/1 | 0/1 | SEQ ID NO: 211 |
| | | | | | | | AATTATTTTTTCTATATATTTTA |
| MD85 | 54899332 | NC_044378.1 | 30801295 | T | 0/0 | 0/1 | SEQ ID NO: 212 |
| | | | | | | | TTATTAAATAAATAGTTTTGACA |
| MD86 | 54904992 | NA | NA | T | 1/1 | 0/1 | SEQ ID NO: 213 |
| | | | | | | | ATAGCCTTGCCTTTACCTACACA |
| MD87 | 54913427 | NA | NA | A | 0/0 | 0/1 | SEQ ID NO: 214 |
| | | | | | | | TGGTAAAATTTTCACACAAGTCA |
| MD88 | 54934549 | NC_044378.1 | 30782136 | C | 1/1 | 0/1 | SEQ ID NO: 215 |
| | | | | | | | AAAAAATTATCGGGTCTCATATA |
| MD89 | 54940067 | NC_044378.1 | 30776608 | C | 1/1 | 0/1 | SEQ ID NO: 216 |
| | | | | | | | GTGCTCGCAAATTCCACTTGCAA |
| MD90 | 54940933 | NC_044378.1 | 30775742 | C | 1/1 | 0/1 | SEQ ID NO: 5 |

“PG Allele” refer to the alleles present in PG_1_19_0125_0002; In Table 2 “MU” refers to “Marker_Upstream” and “MD” refers to “Marker_Downstream” in relation to Marker_0 shown as “M_0”.

### EXAMPLE 2

### Analysis of the THCAS_{G1064A} Marker_0 Polymorphism

### Determination of Expression of the THCAS_{G1064A} Containing Marker_0 SNP

One of the identified SNPs (Marker_0) results in a predicted amino acid change in the resultant protein (FIG. 4). Two possible causes for a loss-of-function gene are either that it is not expressed, or that it contains a mutation that affects the activity of the resultant enzyme. Therefore, primers (SEQ ID NO:217 and SEQ ID NO:218) were designed and made using public information to determine whether THCAS containing the THCAS_{G1064A} polymorphism is expressed in the plant.

RT-PCR analysis showed that THCAS gene expression was similar to that seen in THC producing varieties (FIG. 5).

The primer sequences for the RT-PCR are shown in Table 3 below.

TABLE 3
RT-PCR primer sequences to determine whether
THCAS containing the THCAS_{G1064A} polymorphism
is expressed in the plant.

| Primer ID | Primer sequence | SEQ ID NO |
|---|---|---|
| qRT THCAS F | CAGCAATTCCATTCCCTCAT | SEQ ID NO: 217 |
| qRT THCAS R | TTAGGACTCGCATGATTAGT | SEQ ID NO: 218 |
| | TTTTC | |

### Allelic Discrimination Assay

In the allelic discrimination assay, a KASP (Kompetitive allele specific PCR) marker, KASP03, was designed using the region of THCAS_{G1064A} containing the Marker_0 SNP. Along with a common reverse primer, two forward primers complementary to the sequence with or without the SNP were designed to recognize each form of the allele. Each forward primer, containing a distinct fluorescent label, fluoresces only when incorporated into an amplicon. In a diploid genome the final fluorescent signal generated during a PCR amplification, can discriminate between individuals which are heterozygous, or homozygous for either allele.

Genomic DNA was extracted from Cannabis leaf tissue at seedling stage and the PCR performed. PCR amplifications were performed with the three primers (Table 2) in a Bio-Rad CFX384 Thermal Cycler under the following conditions: An initial activation step for 15 minutes at 94° C.; 9 cycles of denaturation for 20 seconds at 94° C., and annealing/elongation for 60 seconds at 61-55° C. (drop 0.6° C. per cycle)); followed by 25 cycles of denaturation for 20 seconds at 94° C., and annealing/elongation for 60 seconds at 55° C.; and a final read at 30° C. Final fluorescent signals were detected by the thermocycler and analyzed on Bio-Rad CFX Maestro software, which discriminates between individuals heterozygotes or homozygotes for either allele.

Similarly, KASP markers were designed to polymorphisms up- and downstream of the Marker_0 in order to confirm the location of the THCAS_{G1064A} in the genomic region grTHC1.1. KASP14, KASP06, and KASP09, which detect the polymorphisms “Marker_Upstream_123”, “Marker_Upstream_35”, and “Marker_Downstream_90” respectively, were used exactly as described for KASPO3 and all reactions were performed on the same DNA extracted from the same population of plants. The sequences of the KASP molecular markers are provided in Table 4 below. The results of these analyses are summarised in Table 5 and Table 6.

TABLE 4
KASP Primer sequences for detection of Marker_0,
Marker_Upstream_35, Marker Downstream_90 and
Marker_Upstream_123

| Primer ID | Primer sequence | SEQ ID NO |
|---|---|---|
| KASP03_Ref_G_Fwd | AATTAGCAGTGTTAAAATTTACAACACCAC | SEQ ID NO: 219 |
| KASP03_Alt_A_Fwd | AAAATTAGCAGTGTTAAAATTTACAACACCAT | SEQ ID NO: 220 |
| KASP03_Common_Rv | ATTTAGCTGGATTGATACAACCATCTTCTA | SEQ ID NO: 221 |
| KASP06_Ref_T_Fwd | GCCATATGCTGCATCAAAGGCA | SEQ ID NO: 222 |
| KASP06_Alt_C_Fwd | GCCATATGCTGCATCAAAGGCG | SEQ ID NO: 223 |
| KASP06_Common_Rv | CTATGCAGCAAACACCACATACGCTT | SEQ ID NO: 224 |
| KASP09_Ref_C_Fwd | CGCCATTGTTTGTGCTCGCAAAC | SEQ ID NO: 225 |
| KASP09_Alt_T_Fwd | CGCCATTGTTTGTGCTCGCAAAT | SEQ ID NO: 226 |
| KASP09_Common_Rv | CCTCCGCTTCTGATCTTCATTTGCAA | SEQ ID NO: 227 |
| KASP14_Ref_T_Fwd | AGTTGTACGCGTACGTGCCCA | SEQ ID NO: 228 |
| KASP14_Alt_G_Fwd | GTTGTACGCGTACGTGCCCC | SEQ ID NO: 229 |
| KASP14_Common_Rv | AGACAAGCTGTTGGACAATAAGCACTA | SEQ ID NO: 230 |

TABLE 5
KASP marker-based genotype determination of a population of 96 F2 plants produced
from an outcross of PG_1_19_0125_0002. The genotype is shown as either “Alt/Alt”
representing the homozygous allele derived from PG_1_19_0125_0002, or “Ref/Ref”
representing the homozygous allele derived from the high-THCA parent, or heterozygous
“Ref/Alt”. “ND” indicates data points not detected. The chemotype
of each plant is shown as determined by UPLC analysis of leaf material.

| | Chemotype | | | | |
|---|---|---|---|---|---|
| Sample | Dominant | Genotype | | | |
| reference | cannabinoid | KASP03 | KASP06 | KASP09 | KASP14 |
| A1 | THCA | Ref/Ref | Ref/Ref | Ref/Ref | Ref/Ref |
| A10 | THCA | Ref/Alt | Ref/Alt | Ref/Alt | Ref/Alt |
| A11 | THCA | Ref/Alt | Ref/Alt | Ref/Alt | Ref/Alt |
| A12 | THCA | Ref/Alt | Alt/Alt | Ref/Ref | Ref/Alt |
| A2 | CBGA (Low-THCA) | Alt/Alt | Alt/Alt | Alt/Alt | Ref/Alt |
| A3 | CBGA (Low-THCA) | Alt/Alt | Alt/Alt | Alt/Alt | Alt/Alt |
| A4 | THCA | Ref/Ref | Ref/Ref | Ref/Ref | Ref/Ref |
| A5 | THCA | Ref/Alt | Ref/Alt | Ref/Alt | Ref/Alt |
| A6 | THCA | Ref/Ref | Ref/Ref | Ref/Ref | Ref/Ref |
| A7 | THCA | Ref/Alt | Ref/Alt | Ref/Alt | Ref/Alt |
| A8 | THCA | Ref/Ref | Ref/Ref | Ref/Ref | Ref/Alt |
| A9 | THCA | Ref/Alt | Ref/Alt | Ref/Alt | Ref/Alt |
| B1 | THCA | Ref/Alt | Ref/Alt | ND | Ref/Alt |
| B10 | CBGA (Low-THCA) | Alt/Alt | Alt/Alt | Alt/Alt | Alt/Alt |
| B11 | THCA | Ref/Alt | Ref/Alt | Ref/Alt | Ref/Alt |
| B12 | THCA | Ref/Alt | Ref/Alt | Ref/Alt | Ref/Alt |
| B2 | THCA | Ref/Ref | Ref/Ref | Ref/Ref | Ref/Ref |
| B3 | THCA | Ref/Ref | Ref/Ref | Ref/Ref | Ref/Ref |
| B4 | CBGA (Low-THCA) | Alt/Alt | Alt/Alt | Alt/Alt | Ref/Alt |
| B5 | THCA | Ref/Alt | Ref/Alt | Ref/Alt | Ref/Alt |
| B6 | THCA | Ref/Alt | Ref/Ref | Ref/Alt | Ref/Alt |
| B7 | THCA | Ref/Alt | Ref/Alt | Ref/Alt | Ref/Alt |
| B8 | THCA | Ref/Alt | Ref/Alt | Ref/Alt | Ref/Alt |
| B9 | THCA | Ref/Alt | Ref/Alt | Ref/Alt | Ref/Alt |
| C1 | THCA | Ref/Alt | Ref/Alt | Alt/Alt | Ref/Alt |
| C10 | CBGA (Low-THCA) | Alt/Alt | Alt/Alt | Alt/Alt | Ref/Alt |
| C11 | THCA | Ref/Ref | Ref/Ref | Ref/Ref | Ref/Ref |
| C12 | THCA | Ref/Ref | Ref/Ref | Ref/Ref | Ref/Alt |
| C2 | CBGA (Low-THCA) | Alt/Alt | Alt/Alt | Alt/Alt | Ref/Alt |
| C3 | THCA | Ref/Alt | Ref/Alt | Ref/Alt | Ref/Alt |
| C4 | THCA | Ref/Ref | Ref/Ref | Ref/Ref | Ref/Ref |
| C5 | CBGA (Low-THCA) | Alt/Alt | Alt/Alt | Alt/Alt | Ref/Alt |
| C6 | CBGA (Low-THCA) | Alt/Alt | Alt/Alt | Alt/Alt | Ref/Alt |
| C7 | THCA | Ref/Ref | Ref/Ref | Ref/Ref | Ref/Ref |
| C8 | CBGA (Low-THCA) | Alt/Alt | Alt/Alt | Alt/Alt | Alt/Alt |
| C9 | THCA | Ref/Ref | Ref/Ref | Ref/Ref | Ref/Ref |
| D1 | CBGA (Low-THCA) | Alt/Alt | Alt/Alt | Alt/Alt | Ref/Alt |
| D10 | THCA | Ref/Alt | Ref/Alt | Ref/Alt | Ref/Alt |
| D11 | THCA | Ref/Ref | Ref/Ref | Ref/Ref | Ref/Ref |
| D12 | THCA | Ref/Alt | Ref/Alt | Ref/Ref | Ref/Alt |
| D2 | THCA | Ref/Ref | Ref/Ref | ND | Ref/Ref |
| D3 | THCA | Ref/Alt | Ref/Alt | Ref/Alt | Ref/Alt |
| D4 | THCA | Ref/Alt | Ref/Alt | Ref/Alt | Ref/Alt |
| D5 | THCA | Ref/Ref | Ref/Ref | Ref/Ref | Ref/Ref |
| D6 | THCA | Ref/Ref | Ref/Ref | Ref/Ref | Ref/Ref |
| D7 | THCA | Ref/Alt | Ref/Alt | Ref/Alt | Ref/Alt |
| D8 | CBGA (Low-THCA) | Alt/Alt | Alt/Alt | Alt/Alt | Ref/Alt |
| D9 | THCA | Ref/Alt | Ref/Alt | Ref/Alt | Ref/Alt |
| E1 | THCA | Ref/Ref | Ref/Ref | Ref/Alt | Ref/Ref |
| E10 | THCA | Ref/Ref | Ref/Ref | Ref/Ref | Ref/Ref |
| E11 | THCA | Ref/Alt | Ref/Alt | ND | Ref/Alt |
| E12 | CBGA (Low-THCA) | Alt/Alt | Alt/Alt | Ref/Alt | Ref/Alt |
| E2 | CBGA (Low-THCA) | Alt/Alt | Alt/Alt | Alt/Alt | Ref/Alt |
| E3 | THCA | Ref/Alt | Ref/Alt | Ref/Alt | Ref/Alt |
| E4 | THCA | Ref/Alt | Ref/Alt | Ref/Alt | Ref/Alt |
| E5 | THCA | Ref/Alt | Ref/Alt | Ref/Alt | Ref/Alt |
| E6 | THCA | Ref/Ref | Ref/Ref | Ref/Ref | Ref/Ref |
| E7 | THCA | Ref/Alt | Ref/Alt | Ref/Alt | Ref/Alt |
| E8 | THCA | Ref/Ref | Ref/Ref | Ref/Ref | Ref/Ref |
| E9 | THCA | Ref/Alt | Ref/Alt | Ref/Alt | Ref/Alt |
| F1 | THCA | Ref/Ref | Ref/Alt | Alt/Alt | Ref/Alt |
| F10 | THCA | Ref/Alt | Ref/Alt | Ref/Alt | Ref/Alt |
| F11 | CBGA (Low-THCA) | Alt/Alt | Alt/Alt | Alt/Alt | Alt/Alt |
| F12 | CBGA (Low-THCA) | Alt/Alt | Alt/Alt | Alt/Alt | Alt/Alt |
| F2 | CBGA (Low-THCA) | Alt/Alt | Alt/Alt | Alt/Alt | Ref/Alt |
| F3 | THCA | Ref/Alt | Ref/Alt | Ref/Alt | Ref/Alt |
| F4 | THCA | Ref/Alt | Ref/Alt | Ref/Alt | Ref/Alt |
| F5 | THCA | Ref/Ref | Ref/Ref | Ref/Ref | Ref/Ref |
| F6 | CBGA (Low-THCA) | Alt/Alt | Alt/Alt | Alt/Alt | Alt/Alt |
| F7 | THCA | Ref/Alt | Ref/Alt | Ref/Alt | Ref/Alt |
| F8 | THCA | Ref/Alt | Ref/Alt | Ref/Alt | Ref/Alt |
| F9 | THCA | Ref/Alt | Ref/Alt | ND | Ref/Alt |
| G1 | THCA | Ref/Alt | Ref/Ref | Alt/Alt | Ref/Ref |
| G10 | THCA | Ref/Alt | Ref/Alt | Ref/Alt | Ref/Alt |
| G11 | CBGA (Low-THCA) | Alt/Alt | Alt/Alt | Alt/Alt | Ref/Alt |
| G12 | CBGA (Low-THCA) | Alt/Alt | Alt/Alt | Alt/Alt | Ref/Alt |
| G2 | THCA | Ref/Alt | Ref/Alt | Ref/Alt | Ref/Alt |
| G3 | THCA | Ref/Alt | Ref/Alt | Ref/Alt | Ref/Alt |
| G4 | THCA | Ref/Alt | Ref/Alt | Ref/Alt | Ref/Alt |
| G5 | THCA | Ref/Ref | Ref/Ref | Ref/Ref | Ref/Ref |
| G6 | THCA | ND | Ref/Ref | Ref/Ref | Ref/Ref |
| G7 | THCA | Ref/Alt | Ref/Alt | Ref/Alt | Ref/Alt |
| G8 | CBGA (Low-THCA) | Alt/Alt | Alt/Alt | Alt/Alt | Ref/Alt |
| G9 | THCA | Ref/Alt | Ref/Alt | Ref/Alt | Ref/Alt |
| H1 | THCA | Ref/Ref | Ref/Ref | Ref/Alt | Ref/Ref |
| H10 | THCA | Ref/Ref | Ref/Ref | Ref/Ref | Ref/Ref |
| H11 | THCA | Ref/Alt | Ref/Alt | Ref/Alt | Ref/Alt |
| H12 | THCA | Ref/Alt | Ref/Alt | Ref/Alt | Ref/Alt |
| H2 | THCA | Ref/Ref | Alt/Alt | Alt/Alt | Ref/Alt |
| H3 | THCA | Ref/Alt | Ref/Alt | Ref/Alt | Ref/Alt |
| H4 | THCA | Ref/Alt | Ref/Alt | Ref/Alt | Ref/Alt |
| H5 | THCA | Ref/Alt | Ref/Alt | Ref/Alt | Ref/Alt |
| H6 | CBGA (Low-THCA) | Alt/Alt | Alt/Alt | Alt/Alt | Ref/Alt |
| H7 | CBGA (Low-THCA) | Alt/Alt | Alt/Alt | Alt/Alt | Ref/Alt |
| H8 | THCA | Ref/Ref | Ref/Ref | Ref/Ref | Ref/Ref |
| H9 | THCA | Ref/Ref | Ref/Ref | Ref/Ref | Ref/Ref |

TABLE 6
The genotype and chemotype information was used to estimate
the recombination frequency between polymorphisms. The
Marker pairs show the two molecular markers tested. The
Recombination frequency is calculated based on a diploid
genome, where the sum of single or double recombinants
are shown as a percentage of the total potential events.

| | | Potential | |
|---|---|---|---|
| | Recombination | recombination | Recombination |
| Marker pair | events | events | frequency |
| KASP03_KASP06 | 6 | 190 | 3.16 |
| KASP03_KASP09 | 11 | 182 | 6.04 |
| KASP03_KASP14 | 21 | 190 | 11.05 |
| KASP06_KASP09 | 11 | 184 | 5.98 |
| KASP06_KASP14 | 21 | 192 | 10.94 |
| KASP09_KASP14 | 26 | 184 | 14.13 |

### Segregation Analysis

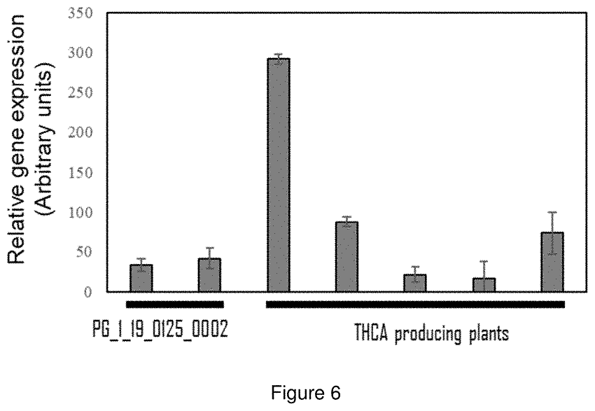

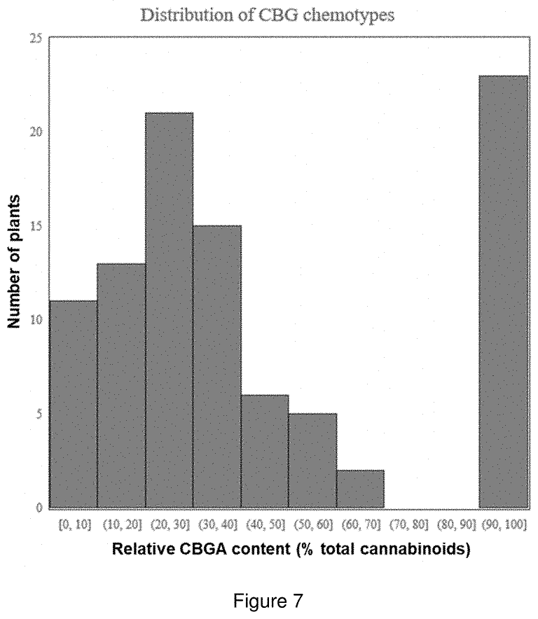

In order to estimate the genetic distance between the THCAS_{G1064A} SNP and the genomic region controlling the low-THC trait a segregating population was generated. The PG_1_19_0125_0002 variety was crossed with a high-THC plant and plants from the resultant the F1 population were selfed. 96 plants of the F2 generation were characterized for both cannabinoid content (FIG. 6) and their DNA extracted for screening with the KASP molecular marker (FIG. 7). If the low-THC phenotype and the SNP are not linked, we expect a random distribution of both in the population with little-to-no correlation. A high correlation can only occur, if the trait of interest is a monogenic and linked. Within the population the trait segregated at 25%, suggesting that it is a single locus, recessive gene inherited in a Mendelian manner, therefore, for the purpose of marker-assisted selection, only a single molecular marker is sufficient for the tracking of the trait and the corresponding genomic region provided the recognized SNP is closely linked to the genetic element controlling the trait.

Comparing the results of the THCA phenotype and the results of the molecular marker in the 96 plants a total correlation was found. The plants homozygous for the Marker_0 SNP presented with a total cannabinoid profile of <5% THCA and >95% CBGA in their total cannabinoid content. In addition, the plants heterozygous for the THCAS_{G1064A} SNP or homozygous for its absence, had a THCA dominant cannabinoid profile.

The perfect correlation across 96 plants shows the tight linkage of the THCAS_{G1064A} SNP and the genetic element controlling for the low-THC phenotype, and provides a distinct and novel utility in a breeding program to select for low-THC varieties. As a molecular marker designed to the THCAS_{G1064A} SNP accurately predicts the THCA phenotype in breeding populations.

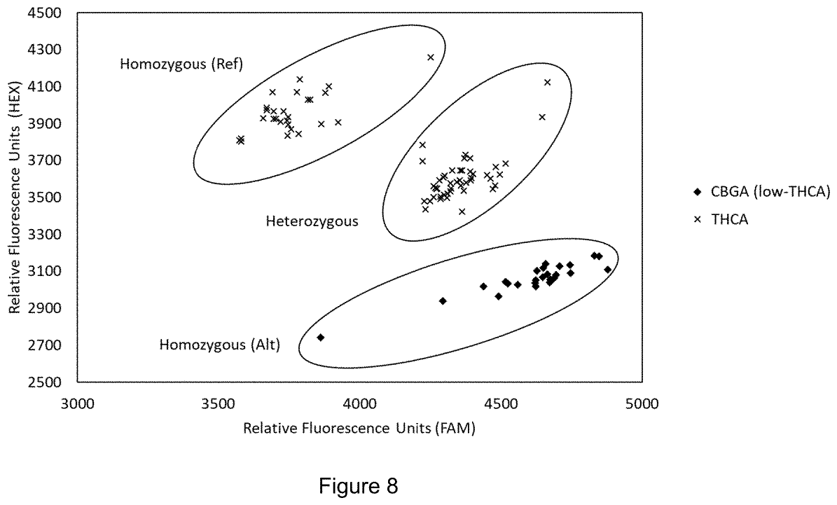

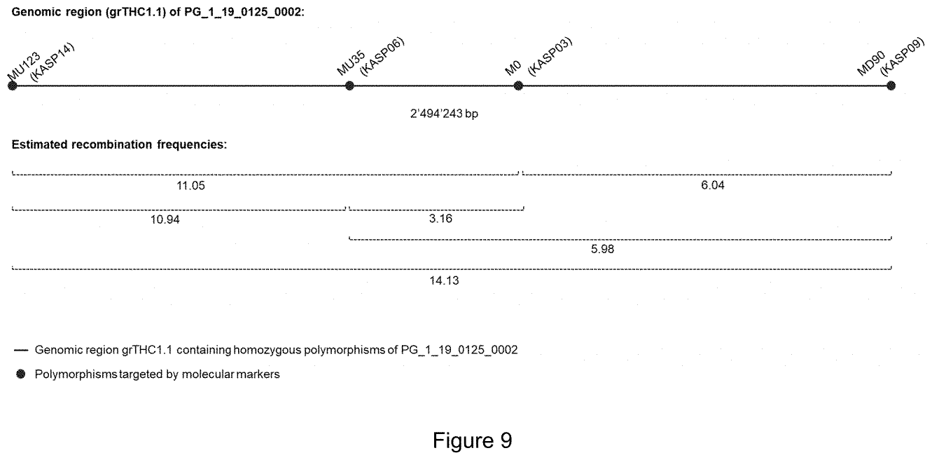

KASP14, KASP06, and KASPO9 which detected “Marker_Upstream_123”, “Marker_Upstream_35”, and “Marker_Downstream_90” respectively, were also tested on the same DNA extracts. Each of these molecular markers correlate well with the chemotype data, but not perfectly. The results are summarised in Table 5. Each reaction that does not correlate with the genotype of Marker_0 represents a recombination event within the genomic region grTHC1.1. The number of recombination events can be used to estimate the recombination frequency between each of the marker pairs (Table 6 and FIG. 8). The most distant marker from Marker_0 is “Marker_Upstream_123” at approximately 1.5 Mb. Here the recombination rate is approximately 11% and so even this marker predicts the low-THCA phenotype with 89% accuracy.

The markers tested here clearly all have utility in the particular cross between the Donor and the Recipient plant described herein. As the recipient plant is a variable, where any Cannabis plant can be used, all 213 markers shown in Table 2 may have utility depending on which Recipient plant is used. In cases where a Recipient plant shares, by chance, some of the polymorphisms of the grTHC1.1 region, others can be used for the design of molecular markers to track the introduction of the genomic region into a novel variety thereby conferring the low-THCA phenotype while retaining the characteristics of the Recipient.

1. An isolated nucleic acid having a nucleotide sequence having a single nucleotide polymorphism associated with low tetrahydrocannabinolic acid (THCA) content in Cannabis sativa, wherein the isolated nucleic acid is selected from the group consisting of:
a) SEQ ID NO: 3;
b) a nucleotide sequence that is 90% identical to SEQ ID NO: 1 and comprises the G1064A single nucleotide polymorphism; and
c) a sequence that is fully complementary to the sequence of (a) or (b).

2. A plant, seed or plant part of Cannabis sativa L., comprising the isolated nucleic acid sequence of claim 1.

3. A method for identifying a Cannabis sativa plant that comprises a low THCA content, the method comprising:
detecting at least one polymorphism in the grTHC1.1 genomic region in a Cannabis sativa plant.

4. The method of claim 3, wherein the at least one polymorphism is selected from the group consisting of the single nucleotide polymorphisms described in SEQ ID NOs:3-216.

5. The method of claim 3, wherein the at least one polymorphism consists of at least one of M0 from SEQ ID NO: 3, MU35 from SEQ ID NO: 4, MD90 from SEQ ID NO: 5, or MU123 from SEQ ID NO: 6.

6. The method of claim 3, wherein the method comprises detecting a haplotype comprising the G1064A SNP from SEQ ID NO: 3 and one or more additional SNPs selected from the marker loci of SEQ ID NOs: 4-216.

7. A Cannabis sativa plant identified by the method of claim 3.

8. A method of producing a Cannabis sativa plant comprising:
introducing one or more single nucleotide polymorphisms (SNPs) selected from the SNPs of SEQ ID NO: 3-216 into a Cannabis sativa plant.

9. The method of claim 8, wherein the THCA content in dry weight (DW) of the mature inflorescence of the Cannabis sativa plant in which the one or more SNPs have been introduced is reduced relative to a Cannabis plant in which the one or more SNPs have not been introduced.

10. The method of claim 8, wherein introducing the one or more SNPs comprises crossing a donor parent plant in which the one or more SNPs is present with a recipient parent plant in which the one or more SNPs is not present.

11. The method of claim 8, wherein introducing the one or more SNPs comprises genetically modifying the Cannabis sativa plant by mutagenesis and/or gene editing.

12. The method of claim 8, wherein the SNP is G1064A.

13. A Cannabis sativa plant produced by the method of claim 8.

14. A method of marker assisted selection comprising screening a population of Cannabis sativa plants, using molecular markers, for plants having at least one allele of a marker locus, wherein the marker locus is the single nucleotide polymorphism (SNP) G1064A in the nucleic acid of SEQ ID NO: 3 or is in linkage disequilibrium with the G1064A SNP and is selected from SEQ ID NO: 4-216;
and selecting a plant comprising the at least one allele.

15. A method of marker assisted breeding comprising:
providing a Cannabis sativa donor parent plant having at least one allele of a marker locus, wherein the marker locus is identified by the method of claim 14 and is associated with low THCA content;
crossing the donor parent plant with a recipient parent plant;
evaluating the progeny for the presence of at least one allele; and
selecting progeny plants having the allele.

16. A progeny Cannabis plant selected by the method of claim 14.

17. An isolated nucleic acid having 90% sequence identity to SEQ ID NO:1, wherein the nucleic acid comprises the single nucleotide polymorphism (SNP) G1064A or C998G.

18. The isolated nucleic acid of claim 17, wherein the nucleic acid encodes a mutant THCAS enzyme having decreased activity compared to a reference THCAS enzyme having the amino acid sequence of SEQ ID NO:232.

19. The nucleic acid of claim 17, wherein the nucleic acid comprises the nucleotide sequence of SEQ ID NO:1.

20. A Cannabis sativa plant comprising a mutant THCAS with 90% sequence identity to SEQ ID NO:1, wherein the nucleic acid comprises the single nucleotide polymorphism (SNP) G1064A or C998G.

21. The Cannabis sativa plant of claim 20, wherein the Cannabis sativa plant has a concentration of less than 0.1% THCA in the dry weight (DW) of the mature inflorescence.

22. A plant extract obtained from a Cannabis sativa plant of claim 20.

23. The plant extract of claim 22 which has a THCA content of less than 0.1%.

24. The plant extract of claim 22, which contains >0.1% THCA, <0.1% CBDA and <0.1% CBCA.

25. The plant extract of claim 22, which contains >0.1% THCA and >1% CBDA and/or >1% CBCA.
